SEQUENCE LISTING (45 sequences)

SEQ ID NO: 1 - protein, 15 aa, Artificial Sequence
  Description of Artificial Sequence Synthetic peptide
IALILEPICCQERAA

SEQ ID NO: 2 - protein, 15 aa, Artificial Sequence
  Description of Artificial Sequence Synthetic peptide
  MOD_RES (1)..(1): Sarcosine
XALILEPICCQERAA

SEQ ID NO: 3 - protein, 15 aa, Artificial Sequence
  Description of Artificial Sequence Synthetic peptide
  MOD_RES (2)..(2): Sarcosine
IXLILEPICCQERAA

SEQ ID NO: 4 - protein, 15 aa, Artificial Sequence
  Description of Artificial Sequence Synthetic peptide
  MOD_RES (3)..(3): Sarcosine
IAXILEPICCQERAA

SEQ ID NO: 5 - protein, 15 aa, Artificial Sequence
  Description of Artificial Sequence Synthetic peptide
  MOD_RES (4)..(4): Sarcosine
IALXLEPICCQERAA

SEQ ID NO: 6 - protein, 15 aa, Artificial Sequence
  Description of Artificial Sequence Synthetic peptide
  MOD_RES (5)..(5): Sarcosine
IALIXEPICCQERAA

SEQ ID NO: 7 - protein, 15 aa, Artificial Sequence
  Description of Artificial Sequence Synthetic peptide
  MOD_RES (6)..(6): Sarcosine
IALILXPICCQERAA

SEQ ID NO: 8 - protein, 15 aa, Artificial Sequence
  Description of Artificial Sequence Synthetic peptide
  MOD_RES (7)..(7): Sarcosine
IALILEXICCQERAA

SEQ ID NO: 9 - protein, 15 aa, Artificial Sequence
  Description of Artificial Sequence Synthetic peptide
  MOD_RES (8)..(8): Sarcosine
IALILEPXCCQERAA

SEQ ID NO: 10 - protein, 15 aa, Artificial Sequence
  Description of Artificial Sequence Synthetic peptide
  MOD_RES (9)..(9): Sarcosine
IALILEPIXCQERAA

SEQ ID NO: 11 - protein, 15 aa, Artificial Sequence
  Description of Artificial Sequence Synthetic peptide
  MOD_RES (10)..(10): Sarcosine
IALILEPICXQERAA

SEQ ID NO: 12 - protein, 14 aa, Artificial Sequence
  Description of Artificial Sequence Synthetic peptide
  MOD_RES (10)..(10): Sarcosine
ALILEPICCXERAA

SEQ ID NO: 13 - protein, 15 aa, Artificial Sequence
  Description of Artificial Sequence Synthetic peptide
  MOD_RES (12)..(12): Sarcosine
IALILEPICCQXRAA

SEQ ID NO: 14 - protein, 15 aa, Artificial Sequence
  Description of Artificial Sequence Synthetic peptide
  MOD_RES (13)..(13): Sarcosine
IALILEPICCQEXAA

SEQ ID NO: 15 - protein, 15 aa, Artificial Sequence
  Description of Artificial Sequence Synthetic peptide
  MOD_RES (14)..(14): Sarcosine
IALILEPICCQERXA

SEQ ID NO: 16 - protein, 15 aa, Artificial Sequence
  Description of Artificial Sequence Synthetic peptide
  MOD_RES (15)..(15): Sarcosine
IALILEPICCQERAX

SEQ ID NO: 17 - protein, 15 aa, Artificial Sequence
  Description of Artificial Sequence Synthetic peptide
  misc_feature (1)..(1): N-term dPEG24
  misc_feature (15)..(15): C-term dPEG24
IALILEPICCQERAA

SEQ ID NO: 18 - protein, 15 aa, Artificial Sequence
  Description of Artificial Sequence Synthetic peptide
  misc_feature (1)..(1): N-term dPEG24
IALILEPICCQERAA

SEQ ID NO: 19 - protein, 15 aa, Artificial Sequence
  Description of Artificial Sequence Synthetic peptide
  misc_feature (15)..(15): C-term dPEG24
IALILEPICCQERAA

SEQ ID NO: 20 - protein, 15 aa, Artificial Sequence
  Description of Artificial Sequence Synthetic peptide
  misc_feature (15)..(15): C-term dPEG20
IALILEPICCQERAA

SEQ ID NO: 21 - protein, 15 aa, Artificial Sequence
  Description of Artificial Sequence Synthetic peptide
  misc_feature (15)..(15): C-term dPEG16
IALILEPICCQERAA

SEQ ID NO: 22 - protein, 15 aa, Artificial Sequence
  Description of Artificial Sequence Synthetic peptide
  misc_feature (15)..(15): C-term dPEG12
IALILEPICCQERAA

SEQ ID NO: 23 - protein, 15 aa, Artificial Sequence
  Description of Artificial Sequence Synthetic peptide
  misc_feature (15)..(15): C-term dPEG08
IALILEPICCQERAA

SEQ ID NO: 24 - protein, 15 aa, Artificial Sequence
  Description of Artificial Sequence Synthetic peptide
  misc_feature (15)..(15): C-term dPEG06
IALILEPICCQERAA

SEQ ID NO: 25 - protein, 15 aa, Artificial Sequence
  Description of Artificial Sequence Synthetic peptide
  misc_feature (15)..(15): C-term dPEG04
IALILEPICCQERAA

SEQ ID NO: 26 - protein, 15 aa, Artificial Sequence
  Description of Artificial Sequence Synthetic peptide
  misc_feature (15)..(15): C-term dPEG03
IALILEPICCQERAA

SEQ ID NO: 27 - protein, 15 aa, Artificial Sequence
  Description of Artificial Sequence Synthetic peptide
  misc_feature (15)..(15): C-term dPEG02
IALILEPICCQERAA

SEQ ID NO: 28 - protein, 15 aa, Artificial Sequence
  Description of Artificial Sequence Synthetic peptide
  MOD_RES (9)..(9): Sarcosine
IALILEPIXAQERAA

SEQ ID NO: 29 - protein, 14 aa, Artificial Sequence
  Description of Artificial Sequence Synthetic peptide
  MOD_RES (9)..(9): Sarcosine
IALILEPIXQERAA

SEQ ID NO: 30 - protein, 15 aa, Artificial Sequence
  Description of Artificial Sequence Synthetic peptide
  MOD_RES (7)..(7): Sarcosine
  MOD_RES (9)..(9): Sarcosine
IALILEXIXCQERAA

SEQ ID NO: 31 - protein, 15 aa, Artificial Sequence
  Description of Artificial Sequence Synthetic peptide
  MOD_RES (6)..(6): Sarcosine
  misc_feature (15)..(15): C-term dPEG24
IALILXPICCQERAA

SEQ ID NO: 32 - protein, 15 aa, Artificial Sequence
  Description of Artificial Sequence Synthetic peptide
  MOD_RES (11)..(11): Sarcosine
  misc_feature (15)..(15): C-term dPEG24
IALILEPICCXERAA

SEQ ID NO: 33 - protein, 15 aa, Artificial Sequence
  Description of Artificial Sequence Synthetic peptide
  MOD_RES (13)..(13): Sarcosine
  misc_feature (15)..(15): C-term dPEG24
IALILEPICCQEXAA

SEQ ID NO: 34 - protein, 15 aa, Artificial Sequence
  Description of Artificial Sequence Synthetic peptide
  MOD_RES (14)..(14): Sarcosine
  misc_feature (15)..(15): C-term dPEG24
IALILEPICCQERXA

SEQ ID NO: 35 - protein, 15 aa, Artificial Sequence
  Description of Artificial Sequence Synthetic peptide
  MOD_RES (6)..(7): Sarcosine
IALILXXICCQERAA

SEQ ID NO: 36 - protein, 15 aa, Artificial Sequence
  Description of Artificial Sequence Synthetic peptide
  MOD_RES (6)..(6): Sarcosine
  MOD_RES (9)..(9): Sarcosine
IALILXPIXCQERAA

SEQ ID NO: 37 - protein, 15 aa, Artificial Sequence
  Description of Artificial Sequence Synthetic peptide
  MOD_RES (7)..(7): Sarcosine
  MOD_RES (11)..(11): Sarcosine
IALILEXICCXERAA

SEQ ID NO: 38 - protein, 15 aa, Artificial Sequence
  Description of Artificial Sequence Synthetic peptide
  MOD_RES (9)..(9): Sarcosine
  MOD_RES (11)..(11): Sarcosine
IALILEPIXCXERAA

SEQ ID NO: 39 - protein, 15 aa, Artificial Sequence
  Description of Artificial Sequence Synthetic peptide
  MOD_RES (7)..(7): Sarcosine
  MOD_RES (13)..(13): Sarcosine
IALILEXICCQEXAA

SEQ ID NO: 40 - protein, 15 aa, Artificial Sequence
  Description of Artificial Sequence Synthetic peptide
  MOD_RES (9)..(9): Sarcosine
  MOD_RES (13)..(13): Sarcosine
IALILEPIXCQEXAA

SEQ ID NO: 41 - protein, 15 aa, Artificial Sequence
  Description of Artificial Sequence Synthetic peptide
  MOD_RES (7)..(7): Sarcosine
  MOD_RES (14)..(14): Sarcosine
IALILEXICCQERXA

SEQ ID NO: 42 - protein, 15 aa, Artificial Sequence
  Description of Artificial Sequence Synthetic peptide
  MOD_RES (9)..(9): Sarcosine
  MOD_RES (14)..(14): Sarcosine
IALILEPIXCQERXA

SEQ ID NO: 43 - protein, 15 aa, Artificial Sequence
  Description of Artificial Sequence Synthetic peptide
  misc_feature (15)..(15): C-term dPEG24
IALILAPICCQARAA

SEQ ID NO: 44 - protein, 15 aa, Artificial Sequence
  Description of Artificial Sequence Synthetic peptide
  MOD_RES (9)..(9): Sarcosine
IALILAPIXCQARAA

SEQ ID NO: 45 - protein, 15 aa, Artificial Sequence
  Description of Artificial Sequence Synthetic peptide
  MOD_RES (7)..(7): Sarcosine
IALILAXICCQARAA

(19) United States
(12) Patent Application Publication
(10) Pub. No.: US 2022/0096592 A1
(43) Pub. Date: Mar. 31, 2022

(54) PIC1 VARIANTS WITH IMPROVED SOLUBILITY AND METHODS OF USING THE SAME

(71) Applicant: REALTA HOLDINGS, LLC, Norfolk, VA (US)

(72) Inventors: Neel K. KRISHNA, Norfolk, VA (US); Kenji CUNNION, Norfolk, VA (US)

(73) Assignee: REALTA HOLDINGS, LLC, Norfolk, VA (US)

(21) Appl. No.: 17/429,111

(22) PCT Filed: Feb. 13, 2020
(86) PCT No.: PCT/US2020/018045

§ 371 Date: Aug. 6, 2021

Related U.S. Application Data
(60) Provisional application No. 62/806,432, filed on Feb. 15, 2019.
(60) Provisional application No. 62/949,181, filed on Dec. 17, 2019.

(51) Int. Cl. A61K 38/10; A61P 37/06

(52) U.S. Cl. CPC A61K 38/10; A61K 35/19; A61K 2300/00; A61P 37/06

(57) ABSTRACT

A method of improving the lifespan of transfused platelets is described. The method may be useful for patients with alloimmunozation who are refractory to transfused platelets. A method of treating delayed hemolytic transfusion reaction is also described. Also described are PIC1 peptide variants with improved solubility and activity.

### CROSS REFERENCE TO RELATED APPLICATION

This application claims the benefit of and priority to United States Provisional Patent Application Nos. 62/806,432 filed on Feb. 15, 2019 and 62/949,181 filed on Dec. 17, 2019, with both applications herein incorporated by reference in their entireties.

### BACKGROUND

Human astroviruses belong to a family of non-enveloped, icosahedral RNA viruses that are an endemic, world-wide pathogen causing acute gastroenteritis in human infants [1]. Unlike calicivirus and rotavirus that cause severe acute disease, astrovirus gastroenteritis is non-inflammatory [2]. The blunted inflammatory response to astrovirus may arise from the 787 amino acid residue astrovirus coat protein that forms the astrovirus capsid. The coat protein was found to inhibit activation of the classical pathway of complement [3]. Complement is an innate immune response of humans that is characterized by a robust inflammatory response to pathogens [4]. Analysis of the amino acid sequence of the astrovirus coat protein identified a region with loose homology to human neutrophil defensin type 1 (HNP-1) and led to development of short peptides that retained the ability to inhibit the classical pathway of complement [5].

Platelet refractoriness continues to be a challenging clinical dilemma for clinicians caring for patients with alloimmunization and development of anti-platelet antibodies [18]. The short lifespan of transfused platelets in these alloimmunized patents greatly limits their ability to maintain platelet levels needed to minimize the risk of bleeding. Current methods of management focus on testing and identifying platelet units less susceptible to the patient's circulating anti-platelet antibodies [24]. These methods are helpful for many patients, but the extra testing is time-consuming and may be only partially successful for individuals with high levels of anti-platelet antibodies or HLA types that are difficult to match or avoid [22]. To date pharmacological interventions have not demonstrated a consistent ability to moderate platelet refractoriness and improve the life span of transfused platelets in alloimmunized patients [24].

Delayed hemolytic transfusion reaction (DHTR) is characterized by a decrease in hematocrit occurring more than one week after the transfusion. For some patients, the drop in hematocrit associated with DHTR can be precipitous, causing severe, even life-threatening, complications. A precipitous drop in hematocrit is sometimes referred to as hyperhemolysis.

The mechanisms underlying DHTR remain poorly understood. The most commonly discussed theory is that DHTRs occur when a patient previously sensitized to a red blood cell (RBC) antigen has undetectable alloantibody levels at the time of transfusion [33]. Thus, the type and cross testing will show no evidence of incompatibility. One to four weeks after transfusion with erythrocytes bearing this antigen, a primary or anamnestic response may occur that causes DHTR [34].

Many DHTRs are believed to be mild and self-limited, such that DHTRs are frequently unidentified. However, a reduction in hemoglobin to the pre-transfusion level between one to two weeks post-transfusion is suspicious for DHTR. Severe DHTR reactions occur without warning and are often life-threatening. They are treated similarly to acute hemolytic transfusion reactions.

In DHTR, it is hypothesized that antibody-coated donor RBCs, usually of the IgG subclass, are destroyed by extravascular hemolysis in the liver and spleen. The major mechanisms of this disease process are believed to occur via Fc-mediated phagocytosis [35]. The role of complement activation in DHTR, if any, remains unknown.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D show sarcosine variant inhibition of complement activation in hemolytic assays and C1q binding. FIG. 1A shows the results of an assay of inhibition of ABO incompatibility hemolysis in a CH50-type assay. Peptides are at a final concentration of 1.8 mM. PIC1 denotes PA-dPEG24. The data shown are the means of n=4 independent experiments+SEM. FIG. 1B shows the results of an assay of inhibition of classical complement pathway-mediated hemolysis in factor B-depleted sera in a CH50-type assay. Peptides are at a final concentration of 0.4 mM. The data shown are the means of n=4 independent experiments+SEM. FIG. 1C shows the results of an ELISA-type assay of binding of increasing concentrations of sarcosine variants to purified C1q. The data shown are the means of n=3 independent experiments±SEM. FIG. 1D shows the results of calculations of half-maximal binding concentrations for each peptide's binding curve.

FIGS. 2A-2D show the results of assays of inhibition of MPO peroxidase activity by sarcosine variants. FIG. 2A shows data in which MPO peroxidase activity was measured in a TMB-based assay for each peptide over a range of concentrations (mM). PIC1 denotes PA-dPEG24. The data shown are the means of n=3 independent experiments±SEM. FIG. 2B is a graph showing the half-maximal inhibition concentrations calculated for each peptide's inhibition curve. FIG. 2C shows the results of an ELISA-type assay of the binding of increasing concentrations of sarcosine variants to purified MPO. The data shown are the means of n=3 independent experiments±SEM. FIG. 2D is a graph showing the half-maximal binding concentrations calculated for each peptide's binding curve.

FIGS. 3A-3H show the results of an assay of sarcosine variant peptides for their ability to protect the MPO heme ring from oxidative degradation. The results are shown for PA-dPEG24 (FIG. 3A), A2 (FIG. 3B), L3 (FIG. 3C), 14 (FIG. 3D), L5 (FIG. 3E), 18 (FIG. 3F), C9 (FIG. 3G) and C9,10 (FIG. 3H). Absorbance spectra for the heme ring is shown in the absence of hydrogen peroxide (MPO), in the presence of hydrogen peroxide (MPO+H₂O₂), in the presence of peptide (MPO+A2), and in the presence of hydrogen peroxide and peptide together (MPO+A2+H₂O₂). Peptides were tested at a concentration of 3.0 mM.

FIG. 4 shows the results of sarcosine variant inhibition of oxidant activity in a Total Antioxidant Capacity (TAC) assay. Antioxidant activity was measured in copper reducing equivalents (CRE). Peptides were tested over a range of concentrations from 0.03-0.25 mM then compared to the standard. The data shown are the means of n=3 independent experiments+SEM.

FIG. 5A is a graph showing the results of an assay of the ability of sarcosine variant peptides to inhibit NET formation. The graph shows sarcosine variant inhibition of free DNA release by neutrophils as a marker of NETosis. Purified human neutrophils were stimulated with 2% normal human sera pre-incubated with ovalbumin-antiovalbumin immune complexes, and 0.05% H₂O₂. Sarcosine peptides and PIC1 were added to the sera to a final concentration of 2 mM. The data shown are the means of n=3 independent experiments+SEM.

FIG. 5B shows the representative fluorescence microscopy images of NET formation after neutrophil stimulation with immune complexes (IC) and hydrogen peroxide (H₂O₂) (second row) compared with neutrophil only control (first row). The third row shows inhibition of NET formation by treating neutrophils stimulated with IC and H₂O₂ with variant 18. Histones are probed with anti-histone antibody (αhistone; left column), neutrophil elastase is probed with anti-neutrophil elastase (αNE; middle column) and DNA is stained with DAPI (right column).

FIGS. 6A-6C illustrate data showing that human platelets sensitized with anti-platelet antibody activate complement decreasing platelet survival. Human platelets were sensitized with anti-platelet antibody (Ab) and incubated with normal human serum (NHS). Increasing concentrations of the classical complement pathway inhibitor PA-dPEG24 (PIC1) was added. FIG. 6A shows the results of an assay in which C3 activation on the platelet surface was measured by iC3b ELISA of stripped platelet membrane bound proteins. The data shown are the means of n=5 independent experiments±SEM. FIG. 6B shows the results of an assay in which generation of the complement anaphylatoxin C5a was measured in the supernatant by C5a ELISA. The data shown are the means of n=4 independent experiments±SEM. In FIG. 6C, cell viability was measured by Presto Blue. The data shown are the means of n=6 independent experiments±SEM.

FIGS. 7A-7C show the results of assays of the viability of human platelets in rat sera. FIG. 7A shows the results of an assay in which human platelets were incubated with increasing amounts of Wistar or Sprague Dawley (S-D) rat sera for 30 minutes, with cell viability was measured with Presto Blue. The data shown are means of n=3 independent experiments±SEM. FIG. 7B shows the results of an assay in which human platelets were incubated with Wistar rat sera (NRS) or heat-inactivated Wistar rat sera (HeatedRS) for increasing amounts of time, with Presto Blue used to assay for viability. The data shown are means of n=3 independent experiments±SEM. FIG. 7C shows the results of an assay in which human platelets were incubated with Wistar rat sera in increasing concentrations of PA-dPEG24 (PIC1). The data shown are means of n=3 independent experiments±SEM.

FIGS. 8A-8C are representative flow cytometry images shown for assays of human platelets stained with PKH26. In FIG. 8A, unstained human platelets assayed by flow cytometry. In FIG. 8B, human platelets stained with PKH26 were assayed by flow cytometry. In FIG. 8C, stained and unstained human platelets were mixed in a 1:1 ratio, and assayed by flow cytometry.

FIGS. 9A-9C are representative flow cytometry images shown for assays of PKH26-stained human platelets transfused into a Wistar rat. In FIG. 9A, a pre-transfusion blood sample (T=0) was assayed by flow cytometry. In FIG. 9B, a blood sample drawn 0.5 minutes after platelet transfusion (T=0.5) was assayed by flow cytometry. In FIG. 9C, blood drawn two minutes after platelet transfusion was assayed by flow cytometry.

FIG. 10 shows histological visualization of transfused PKH26 stained platelets via fluorescence microscopy. PKH26 stained platelets were transfused (top row). Rats were sham transfused (bottom row). Representative fluorescence microscopy images of the liver and spleen are shown.

FIG. 11 shows the results of a flow cytometry assay of PKH26-stained human platelets transfused into Wistar rats, with or without PA-dPEG24 (PIC1). PA-dPEG24 (160 mg/kg) was infused 30 seconds prior to transfusion with stained human platelets. Rats received a transfusion of 1×10⁸ stained human platelets. Blood samples were drawn pre-transfusion (T=0), at 0.5 minutes after platelet transfusion (T=0.5) or 2 minutes (T=2) after platelet transfusion. Representative flow cytometry images are shown.

FIGS. 12A-12B show the results of an in vivo platelet survival assay in the presence of complement inhibition with PA-dPEG24. PA-dPEG24 (PIC1) at 160 mg/kg was infused 30 seconds prior to platelet transfusion. In FIG. 12A, PKH26 stained human platelets (1×10⁸) were transfused and blood draws were obtained over time. The percentage of stained human platelets relative to the total number of platelets in the blood sample was assayed by flow cytometry. The data shown are the means of n=4 rats in each group±SEM. In FIG. 12B, unstained human platelets (1×108) were transfused and blood draws were obtained over time. The total number of platelets in the blood samples was measured by a commercial vendor. The change in total platelet count over time is shown relative to the absolute platelet count for each rat 0.5 minutes after platelet transfusion. The data shown are the mean for n=4 rats in each group±SEM.

FIG. 13 is a graph showing the degree of hemolysis of erythrocytes in a subject's plasma in a complement-permissive buffer in the presence of increasing concentrations of PIC1 (PA-dPEG24) The data show means of (n=3) independent experiments and the standard error of the mean (SEM).

### SUMMARY OF THE INVENTION

In one aspect is provided a method for inhibiting an immune response to transfused platelets in a subject, the method comprising the steps of: a) administering a classical complement pathway inhibitor to a subject in need thereof, and b) transfusing platelets to the subject.

In another aspect is provided a method for inhibiting refractoriness to platelets in an alloimmunized subject, the method comprising the steps of: a) treating platelets with a classical complement pathway inhibitor; and b) transfusing the treated platelets to the subject

In another aspect is provided a method of preventing platelet refractoriness in a subject receiving platelets from an antigenically mismatched donor, the method comprising administering a classical complement pathway inhibitor to the subject before the platelets are transfused to the subject.

In another aspect is provided a method of preventing platelet refractoriness in a subject receiving platelets from an antigenically mismatched donor, the method comprising the steps of: a) treating platelets with a classical complement pathway inhibitor; and b) transfusing the treated platelets to the subject.

In various embodiments of the above aspects, the method is effective to increase survival of the transfused platelets in the subject. In various embodiments, the method is effective to reduce complement-mediated attack of the transfused platelets in the subject. In various embodiments, the method is effective to increase survival of the transfused platelets in the subject.

In some embodiments, the subject is human.

In some embodiments, the complement mediated inhibitor is a PIC1 peptide. In some embodiments, the PIC1 peptide comprises an amino acid sequence at least 85% identical to any one of SEQ ID NOS: 1-45. In certain embodiments, the PIC1 peptide comprises an amino acid sequence at least 85% identical to any one of SEQ ID NOS: 3, 4, 5, 6, 9, 10, 19, and 29. In certain embodiments, the PIC1 peptide comprises the amino acid sequence of any one of SEQ ID NOS: 3, 4, 5, 6, 9, 10, 19, and 29. In certain embodiments, the PIC1 peptide comprises the amino acid sequence of SEQ ID NO: 3. In certain embodiments, the PIC1 peptide comprises the amino acid sequence of SEQ ID NO: 4. In certain embodiments, the PIC1 peptide comprises the amino acid sequence of SEQ ID NO: 5. In certain embodiments, the PIC1 peptide comprises the amino acid sequence of SEQ ID NO: 6. In certain embodiments, the PIC1 peptide comprises the amino acid sequence of SEQ ID NO: 9. In certain embodiments, the PIC1 peptide comprises the amino acid sequence of SEQ ID NO: 10. In certain embodiments, the PIC1 peptide comprises the amino acid sequence of SEQ ID NO: 19. In certain embodiments, the PIC1 peptide comprises the amino acid sequence of SEQ ID NO: 29.

In another aspect is provided a blood product for transfusion into a recipient, where the blood product comprises platelets and a classical complement pathway inhibitor. In some embodiments, the complement mediated inhibitor is a PIC1 peptide. In some embodiments, the PIC1 peptide comprises an amino acid sequence at least 85% identical to any one of SEQ ID NOS: 3, 4, 5, 6, 9, 10, 19, and 29. In some embodiments, the PIC1 peptide comprises the amino acid sequence of any one of SEQ ID NOS: 3, 4, 5, 6, 9, 10, 19, and 29. In certain embodiments, the PIC1 peptide comprises the amino acid sequence of SEQ ID NO: 3. In certain embodiments, the PIC1 peptide comprises the amino acid sequence of SEQ ID NO: 4. In certain embodiments, the PIC1 peptide comprises the amino acid sequence of SEQ ID NO: 5. In certain embodiments, the PIC1 peptide comprises the amino acid sequence of SEQ ID NO: 6. In certain embodiments, the PIC1 peptide comprises the amino acid sequence of SEQ ID NO: 9. In certain embodiments, the PIC1 peptide comprises the amino acid sequence of SEQ ID NO: 10. In certain embodiments, the PIC1 peptide comprises the amino acid sequence of SEQ ID NO: 19. In certain embodiments, the PIC1 peptide comprises the amino acid sequence of SEQ ID NO: 29.

In another aspect is provided a method of treating delayed hemolytic transfusion reaction (DHTR) in a subject. The method comprises administering a therapeutically effective amount of a classical complement pathway inhibitor to the subject.

In some embodiments, the classical complement pathway inhibitor is administered parenterally. In some embodiments, the classical complement pathway inhibitor is administered intravenously. In various embodiments, the subject is human. In some embodiments, the complement mediated inhibitor is a PIC1 peptide. In some embodiments, the PIC1 peptide comprises an amino acid sequence at least 85% identical to any one of SEQ ID NOS: 3, 4, 5, 6, 9, 10, 19, and 29. In some embodiments, the PIC1 peptide comprises the amino acid sequence of any one of SEQ ID NOS: 3, 4, 5, 6, 9, 10, 19, and 29. In certain embodiments, the PIC1 peptide comprises the amino acid sequence of SEQ ID NO: 3. In certain embodiments, the PIC1 peptide comprises the amino acid sequence of SEQ ID NO: 4. In certain embodiments, the PIC1 peptide comprises the amino acid sequence of SEQ ID NO: 5. In certain embodiments, the PIC1 peptide comprises the amino acid sequence of SEQ ID NO: 6. In certain embodiments, the PIC1 peptide comprises the amino acid sequence of SEQ ID NO: 9. In certain embodiments; the PIC1 peptide comprises the amino acid sequence of SEQ ID NO: 10. In certain embodiments, the PIC1 peptide comprises the amino acid sequence of SEQ ID NO: 19. In certain embodiments, the PIC1 peptide comprises the amino acid sequence of SEQ ID NO: 29.

### DETAILED DESCRIPTION OF THE INVENTION

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below.

The term “inhibition” refers to the reduction in the biological function of an enzyme, protein, peptide, factor, byproduct, or derivative thereof either individually or in complexes; reduction in the quantity of a biological protein, peptide, or derivative thereof whether in vivo or in vitro; or interruption of a biological chain of events, cascade, or pathway known to comprise a related series of biological or chemical reactions. The term “inhibition” may thus be used, for example, to describe the reduction of quantity of a single component of the complement cascade compared to a control sample, a reduction in the rate or total amount of formation of a component or complex of components, or the reduction of the overall activity of a complex process or series of biological reactions leading to such outcomes as cell lysis, formation of convertase enzymes, formation of complement-derived membrane attack complexes, inflammation, or inflammatory disease. In an in vitro assay, the term “inhibition” may refer to the measurable reduction of some biological or chemical event, but the person of ordinary skill in the art will appreciate that the measurable reduction need not be total to be “inhibitory.”

The term “PIC1” refers to a peptide comprising the polar assortant (PA) sequence of IALILEPICCQERAA (SEQ ID NO: 1), as well as peptides comprising the same amino acid sequence but with modifications such as PEGylation. The term “PIC1 variant” refers to peptides comprising a sequence that is at least 85% identical, or at least 90% identical, or at least 95% identical, or at least 99% identical, but not 100% identical, to the PA sequence of IALILEPICCQERAA (SEQ ID NO: 1). PIC1 variants may comprise peptides with at least one of the amino acids of the PA sequence deleted. PIC1 variants may comprise peptides with an amino acid inserted into the PA sequence. PIC1 variants may comprise peptides with at least one of the amino acids of the PA sequence substituted with another amino acid, such as alanine, a modified amino acid or an amino acid derivative, such as sarcosine (Sar).

The term “subject” as used herein means any subject for whom diagnosis, prognosis, or therapy is desired. For example, a subject can be a mammal, e.g., a human or non-human primate (such as an ape, monkey, orangutan, or chimpanzee), a dog, cat, guinea pig, rabbit, rat, mouse, horse, cattle, or cow.

The term “therapeutically effective amount” as used herein refers to the total amount of each active component that is sufficient to show a meaningful patient benefit. The therapeutically effective amount of the peptide compound varies depending on several factors, such as the condition being treated, the severity of the condition, the time of administration, the route of administration, the rate of excretion of the compound employed, the duration of treatment, the co-therapy involved, and the age, gender, weight, and condition of the subject, etc. One of ordinary skill in the art can determine the therapeutically effective amount. Accordingly, one of ordinary skill in the art may need to titer the dosage and modify the route of administration to obtain the maximal therapeutic effect.

As used herein, “treat,” “treating,” or “treatment” refers to administering a therapy in an amount, manner (e.g., schedule of administration), and/or mode (e.g., route of administration), effective to improve a disorder (e.g., a disorder described herein) or a symptom thereof, or to prevent or slow the progression of a disorder (e.g., a disorder described herein) or a symptom thereof. This can be evidenced by, e.g., an improvement in a parameter associated with a disorder or a symptom thereof, e.g., to a statistically significant degree or to a degree detectable to one skilled in the art. An effective amount, manner, or mode can vary depending on the subject and may be tailored to the subject. By preventing or slowing progression of a disorder or a symptom thereof, a treatment can prevent or slow deterioration resulting from a disorder or a symptom thereof in an affected or diagnosed subject.

In one aspect is provided a method of inhibiting inflammation in a subject comprising administering a therapeutically effective amount of PIC1, or a PIC1 variant, to the subject. In another aspect is provided a method of treating an inflammatory disorder in a subject comprising administering a therapeutically effective amount of PIC1, or a PIC1 variant, to the subject. In a related aspect is provided a PIC1 or PIC1 variant for use in a method of treating and/or preventing inflammation in a subject. The method comprises administering to the subject in need thereof a composition comprising a therapeutically effective amount of the PIC1 or the PIC1 variant.

Examples of PIC1 and PIC1 variants include, but are not limited to, the peptides listed in Table 1.

TABLE 1

| Peptide name | Peptide sequence |
|---|---|
| PA | IALILEPICCQERAA |
| | (SEQ ID NO: 1) |
| PA-I1Sar | (Sar)ALILEPICCQERAA |
| | (SEQ ID NO: 2) |
| PA-A2Sar | I(Sar)LILEPICCQERAA |
| | (SEQ ID NO: 3) |
| PA-L3Sar | IA(Sar)ILEPICCQERAA |
| | (SEQ ID NO: 4) |
| PA-I4Sar | IAL(Sar)LEPICCQERAA |
| | (SEQ ID NO: 5) |
| PA-L5Sar | IALI(Sar)EPICCQERAA |
| | (SEQ ID NO: 6) |
| PA-E6Sar | IALIL(Sar)PICCQERAA |
| | (SEQ ID NO: 7) |
| PA-P7Sar | IALILE(Sar)ICCQERAA |
| | (SEQ ID NO: 8) |
| PA-I8Sar | IALILEP(Sar)CCQERAA |
| | (SEQ ID NO: 9) |
| PA-C9Sar | IALILEPI(Sar)CQERAA |
| | (SEQ ID NO: 10) |
| PA-C10Sar | IALILEPIC(Sar)QERAA |
| | (SEQ ID NO: 11) |
| PA-Q11Sar | ALILEPICC(Sar)ERAA |
| | (SEQ ID NO: 12) |
| PA-E12Sar | IALILEPICCQ(Sar)RAA |
| | (SEQ ID NO: 13) |
| PA-R13Sar | IALILEPICCQE(Sar)AA |
| | (SEQ ID NO: 14) |
| PA-A14Sar | IALILEPICCQER(Sar)A |
| | (SEQ ID NO: 15) |
| PA-A15Sar | IALILEPICCQERA(Sar) |
| | (SEQ ID NO: 16) |
| dPEG24-PA-dPEG24 | dPEG24-IALILEPICCQERAA-dPEG24 |
| | (SEQ ID NO: 17) |
| dPEG24-PA | dPEG24-IALILEPICCQERAA |
| | (SEQ ID NO: 18) |
| PA-dPEG24 | IALILEPICCQERAA-dPEG24 |
| | (SEQ ID NO: 19) |
| PA-dPEG20 | IALILEPICCQERAA-dPEG20 |
| | (SEQ ID NO: 20) |
| PA-dPEG16 | IALILEPICCQERAA-dPEG16 |
| | (SEQ ID NO: 21) |
| PA-dPEG12 | IALILEPICCQERAA-dPEG12 |
| | (SEQ ID NO: 22) |
| PA-dPEG08 | IALILEPICCQERAA-dPEG08 |
| | (SEQ ID NO: 23) |
| PA-dPEGO6 | IALILEPICCQERAA-dPEG06 |
| | (SEQ ID NO: 24) |
| PA-dPEG04 | IALILEPICCQERAA-dPEG04 |
| | (SEQ ID NO: 25) |
| PA-dPEG03 | IALILEPICCQERAA-dPEG03 |
| | (SEQ ID NO: 26) |
| PA-dPEG02 | IALILEPICCQERAA-dPEG02 |
| | (SEQ ID NO: 27) |
| PA-C9SarC10A | IALILEPI(Sar)AQERAA |
| | (SEQ ID NO: 28) |
| PA-C9SarD10 | IALILEPI(Sar)QERAA |
| | (SEQ ID NO: 29) |
| PA-P7SarC9Sar | IALILE(Sar)I(Sar)CQERAA |
| | (SEQ ID NO: 30) |
| PA-E6Sar-dPEG24 | IALIL(Sar)PICCQERAA-dPEG24 |
| | (SEQ ID NO: 31) |
| PA-Q11Sar-dPEG24 | IALILEPICC(Sar)ERAA-dPEG24 |
| | (SEQ ID NO: 32) |
| PA-R13Sar-dPEG24 | IALILEPICCQE(Sar)AA-dPEG24 |
| | (SEQ ID NO: 33) |
| PA-A14Sar-dPEG24 | IALILEPICCQER(Sar)A-dPEG24 |
| | (SEQ ID NO: 34) |
| E6SarP7Sar | IALIL(Sar)(Sar)ICCQERAA |
| | (SEQ ID NO: 35) |
| E6SarC9Sar | IALIL(Sar)PI(Sar)CQERAA |
| | (SEQ ID NO: 36) |
| Q11SarP7Sar | IALILE(Sar)ICC(Sar)ERAA |
| | (SEQ ID NO: 37) |
| Q11SarC9Sar | IALILEPI(Sar)C(Sar)ERAA |
| | (SEQ ID NO: 38) |
| R13SarP7Sar | IALILE(Sar)ICCQE(Sar)AA |
| | (SEQ ID NO: 39) |
| R13SarC9Sar | IALILEPI(Sar)CQE(Sar)AA |
| | (SEQ ID NO: 40) |
| A14SarP7Sar | IALILE(Sar)ICCQER(Sar)A |
| | (SEQ ID NO: 41) |
| A14SarC9Sar | IALILEPI(Sar)CQER(Sar)A |
| | (SEQ ID NO: 42) |
| E6AE12A-dPEG24 | IALILAPICCQARAA-dPEG24 |
| | (SEQ ID NO: 43) |
| E6AE12AC9Sar | IALILAPI(Sar)CQARAA |
| | (SEQ ID NO: 44) |
| E6AE12AP7Sar | IALILA(Sar)ICCQARAA |
| | (SEQ ID NO: 45) |

In some embodiments, PIC1 comprises one or more PEG moieties. The PEG moieties may be attached to the N-terminus, the C-terminus, or both the N-terminus and C-terminus by PEGylation. In one or more embodiments, 24 PEG moieties are attached to the N-terminus. In one or more embodiments, 24 PEG moieties are attached to the C-terminus. In one or more embodiments, 24 PEG moieties are attached to the N-terminus and to the C-terminus. In one or more embodiments, 1, 2, 3, 4, 5, 6, 7, 8, 9, 10, 11, 12, 13, 14, 15, 16, 17, 18, 19, 20, 21, 22, 23, or 24 PEG moieties are attached to the N-terminus. In one or more embodiments, 1, 2, 3, 4, 5, 6, 7, 8, 9, 10, 11, 12, 13, 14, 15, 16, 17, 18, 19, 20, 21, 22, 23, or 24 PEG moieties are attached to the C-terminus. In one or more embodiments, 1, 2, 3, 4, 5, 6, 7, 8, 9, 10, 11, 12, 13, 14, 15, 16, 17, 18, 19, 20, 21, 22, 23, or 24 PEG moieties are attached to both the N-terminus and the C-terminus.

The PIC1 peptide may be a synthetic peptide. A synthetic peptide is prepared in vitro. Synthetic peptides can be prepared according to various methods known in the art. For example, a synthetic peptide can be prepared by sequentially coupling individual amino acids to form the peptide. In some embodiments, the carboxyl group of individual amino acids is sequentially coupled to the amino terminus of a growing peptide chain. Protecting groups can be used to prevent unwanted side reactions from occurring during the coupling process. Peptide synthesis can occur in liquid phase or in solid phase.

Exemplary PIC1 peptides include, but are not limited to, PA-dPEG24 (a peptide comprising the polar assortant (PA) sequence and 24 PEG moieties at the C-terminus), PA-dPEG20 (comprising 20 PEG moieties at the C-terminus), PA-dPEG16 (comprising 16 PEG moieties at the C-terminus), PA-dPEG12 (comprising 12 PEG moieties at the C-terminus), PA-dPEG08 (comprising 8 PEG moieties at the C-terminus), PA-dPEG06 (comprising 6 PEG moieties at the C-terminus), PA-dPEG04 (comprising 4 PEG moieties at the C-terminus), PA-dPEG03 (comprising 3 PEG moieties at the C-terminus), and PA-dPEG02 (comprising 2 PEG moieties at the C-terminus).

PIC1 peptides can inhibit the classical pathway of complement by binding and blocking activation of the initiating component of the cascade, C1. PA-dPEG24 is a 15-amino acid PEGylated peptide in the PIC1 family. pA-dPEG24 comprises the sequence of SEQ ID NO: 19. PA-dPEG24 can inhibit immune complex-initiated complement activation as well as inhibit NET formation. PA-dPEG24 can consistently inhibit complement activation by a variety of immune complexes and can also inhibit NET formation initiated by several stimuli.

Described herein are sarcosine substitution variants that were soluble in water without requiring PEGylation. These sarcosine substitution variants include peptides comprising an amino acid sequence of any one of SEQ ID NOS: 3, 4, 5, 6, 9, 10 and 29. The examples provided herein show that substitution with sarcosine of the isoleucine at position eight yielded a soluble peptide that surpassed the parent molecule for complement inhibition and myeloperoxidase inhibition. Substitution with sarcosine of the cysteine at position nine improved solubility, but did not otherwise change the functional characteristics compared with the parent compound. However, replacement of both vicinal cysteine residues at positions 9 and 10 with a single sarcosine residue reduced functional activity in most of the assays tested.

Several of the sarcosine PIC1 variants described herein have improved solubility as well as a number of unanticipated structure-function findings that provide new insights. Several sarcosine substitution variants demonstrate increased potency over the parent peptide, which may provide for increased therapeutic potential for inflammatory disease processes involving complement, myeloperoxidase, NETs or oxidant stress.

Without wishing to be bound by theory, sarcosine residues are frequently used in medicinal chemistry due to the favorable solubility profile, reduction in the number of intra- or inter-molecular hydrogen bonds due to absence of the proton from the NH group and potential alteration of neighboring residues due to changes in the ϕ, ψ torsion angles resulting in increased steric constraints [16]. Sarcosine substitution of the relatively hydrophobic alanine, leucine and isoleucine amino acids at positions 2-5 and position 8 can greatly improve solubility by decreasing the overall hydrophobicity of the peptide. Unexpectedly, data provided herein shows that sarcosine substitution of cysteine at position 9, but not at position 10, also improved solubility as did substitution of both vicinal cysteines (C9,10) with a single sarcosine. Without wishing to be bound by theory, cysteine residues are uncharged and relatively polar. The observation that solubility is enhanced with sarcosine substitution at position 9, both positions 9 and 10, but not at position 10, suggests that solubility can be affected by altering the conformation of the peptide rather than just by modifying overall hydrophobicity.

Also described herein is a method for inhibiting an immune response to transfused platelets in a subject, said method comprising the steps of: a) administering a classical complement pathway inhibitor to a subject in need thereof; and b) transfusing platelets to the subject. The classical complement pathway inhibitor can be a peptide comprising the sequence of any of SEQ ID NOS: 1-45, e.g., peptides comprising the sequence of any of SEQ ID NOS: 3, 4, 5, 6, 9, 10, 19 and 29. The complement pathway inhibitor may be administered before the transfusion, during the transfusion (e.g., as part of the transfused platelets), or after the transfusion. The complement pathway inhibitor may be administered one hour before the transfusion, 20-40 minutes before the transfusion, 10-20 minutes before the transfusion, 1-10 minutes before the transfusion, about one minute before the transfusion, one minute before the transfusion, or less than one minute before the transfusion. The complement pathway inhibitor may be administered one hour after the transfusion, 20-40 minutes after the transfusion, 10-20 minutes after the transfusion, 1-10 minutes after the transfusion, about one minute after the transfusion, one minute after the transfusion, or less than one minute after the transfusion.

Also described herein is a method for inhibiting refractoriness to platelets in an alloimmunized subject, comprising the steps of: a) treating platelets with a classical complement pathway inhibitor; and b) transfusing the treated platelets to the subject. The classical complement pathway inhibitor can be a peptide comprising the sequence of any of SEQ ID NOS: 1-45, e.g., peptides comprising the sequence of any of SEQ ID NOS: 3, 4, 5, 6, 9, 10, 19 and 29.

Also described herein is a method of preventing platelet refractoriness in a subject receiving platelets from an antigenically mismatched donor, the method comprising administering a classical complement pathway inhibitor to the subject before the platelets are transfused to the subject. The classical complement pathway inhibitor can be a peptide comprising the sequence of any of SEQ ID NOS: 1-45, e.g., peptides comprising the sequence of any of SEQ ID NOS: 3, 4, 5, 6, 9, 10, 19 and 29. The complement pathway inhibitor may be administered one hour before the transfusion, 20-40 minutes before the transfusion, 10-20 minutes before the transfusion, 1-10 minutes before the transfusion, about one minute before the transfusion, one minute before the transfusion, or less than one minute before the transfusion.

Also described herein is a method of preventing platelet refractoriness in a subject receiving platelets from an antigenically mismatched donor, the method comprising the steps of: a) treating platelets with a classical complement pathway inhibitor; and b) transfusing the treated platelets to the subject.

In experiments described herein, an ex vivo system is used to demonstrate that human platelets sensitized with anti-platelet antibodies initiate complement activation, resulting in decreased cell viability. It is shown that most of the sensitized platelet initiated complement activation occurs via the classical pathway. Additionally, classical pathway inhibitors can protect antibody-sensitized platelets from human sera complement-mediated killing.

Wistar rat sera contain natural antibodies that initiate complement-mediated lysis of human A or AB type erythrocytes [31]. Without wishing to be bound by theory, Wistar rat sera may also cause complement-mediated killing of human platelets, demonstrating that human platelets are incompatible in Wistar rat sera. Complement-mediated destruction of human platelets via Wistar rat sera occurs largely via the classical pathway. Described herein is a new animal model of platelet refractoriness utilizing human platelets transfused into Wistar rats. PKH26-stained human platelets transfused into Wistar rats were measurable by flow cytometry, though such platelets have a short circulating half-life consistent with immune incompatibility. A classical pathway complement inhibitor, PA-dPEG24, was effective to briefly increase the numbers of circulating incompatible platelets.

In one aspect is provided a method of treating DHTR in a subject comprising administering a therapeutically effective amount of PIC1, or a PIC1 variant, to the subject. The method comprises administering to the subject in need thereof a composition comprising a therapeutically effective amount of the PIC1 or the PIC1 variant.

Described herein for the first time is the use of a complement inhibitor can treat, ameliorate or otherwise moderate the progression of DHTR. Without wishing to be bound by theory, hemolysis in DHTR may involve antibody-Fc receptor interactions that lead to phagocytosis [35]. PIC1 peptides can inhibit the classical pathway of complement by binding and blocking activation of the initiating component of the cascade, C1. One exemplary PIC1 peptide is PA-dPEG24, a 15-amino acid PEGylated peptide. PA-dPEG24 can consistently inhibit complement activation by a variety of immune complexes. PA-dPEG24 can also inhibit NET formation initiated by several stimuli.

In a related aspect is provided a PIC1 or PIC1 variant for use in a method of treating and/or preventing DHTR in a subject. The method comprises administering to the subject in need thereof a composition comprising a therapeutically effective amount of the PIC1 or the PIC1 variant.

In some embodiments, the PIC1 is administered after the subject is administered the blood transfusion and before any symptoms of DHTR.

Without wishing to be bound by theory, antibody-mediated activation of the complement system is directed by the classical complement pathway in which the initiating complex, C1, is bound by IgM or multiple IgG triggering activation and downstream effector functions (i.e., C3a, C5a and membrane attack complex formation). PIC1 peptide inhibitors of the classical complement pathway can bind C1q, the recognition molecule of the C1 complex, to prevent antibody-mediated activation. PA-dPEG24 (IALILEPICCQERAA-dPEG24 (SEQ ID NO: 19)) has been demonstrated to inhibit classical pathway activation both in vitro and in vivo when administered intravascularly into rats where it can achieve >90% systemic inhibition of complement activation by 30 seconds.

In some embodiments, a classical complement pathway inhibitor is used to treat active hemolysis in a patient suffering a delayed hemolytic transfusion reaction.

### Examples

The present invention is also described and demonstrated by way of the following examples. However, the use of this and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

The following Materials and Methods section applies to Examples 1-6 below. In these examples, sarcosine substitution variants were synthesized, evaluated for solubility in water, and then tested in standard complement, myeloperoxidase, NET formation and antioxidant capacity assays.

### Materials and Methods for Examples 1-6

Blood from healthy donors was obtained with written consent under an Eastern Virginia Medical School IRB approved protocol, 02-06-EX 0216. Blood was used for the preparation of reagents: purified platelets, erythrocytes and neutrophils.

### Reagents

PA-dPEG24 (IALILEPICCQERAA-dPEG24 or PIC1) was manufactured by PolyPeptide Group (San Diego, Calif.) to ≥95% purity verified by HPLC and mass spectrometry analysis. Lyophilized PA-dPEG24 was solubilized in 0.05 M Histidine buffer and pH adjusted to 6.7. Sarcosine substitution derivative peptides and the base peptide IALILEPICCQERAA (PA) were synthesized by New England Peptide (Gardner, M A) to >90% purity. Sarcosine variants and PEG were dissolved in water and the pH was adjusted with NaOH. PA was dissolved in DMSO and then brought up to the final concentration with water resulting in 30% DMSO and pH adjusted. Antibody sensitized sheep erythrocytes (EA), purified C1q and factor B-depleted human sera were purchased from Complement Technology (Tyler, Tex.). Purified myeloperoxidase was purchased from Lee BioSolutions (Maryland Heights, Mo.) and tetramethylbenzidine (TMB) and PicoGreen were purchased from Thermo Fisher (Waltham Mass.).

The peptides shown in Table 2, including sixteen sarcosine substitution variants, were synthesized.

TABLE 2
Peptide designations and sequences.

| | | SEQ |
|---|---|---|
| Name | Sequence | ID NO: |
| PA-dPEG24 | H2N-IALILEPICCQERAA-dPEG24 | 19 |
| (PIC1) | | |
| PA | H2N-IALILEPICCQERAA-OH | 1 |
| I1 | H2N-(Sar)ALILEPICCQERAA-OH | 2 |
| A2 | H2N-I(Sar)LILEPICCQERAA-OH | 3 |
| L3 | H2N-IA(Sar)ILEPICCQERAA-OH | 4 |
| I4 | H2N-IAL(Sar)LEPICCQERAA-OH | 5 |
| L5 | H2N-IALI(Sar)EPICCQERAA-OH | 6 |
| E6 | H2N-IALIL(Sar)PICCQERAA-OH | 7 |
| P7 | H2N-IALILE(Sar)ICCQERAA-OH | 8 |
| I8 | H2N-IALILEP(Sar)CCQERAA-OH | 9 |
| C9 | H2N-IALILEPI(Sar)CQERAA-OH | 10 |
| C10 | H2N-IALILEPIC(Sar)QERAA-OH | 11 |
| C9, 10 | H2N-IALILEPI(Sar)QERAA-OH | 29 |
| Q11 | H2N-IALILEPICC(Sar)ERAA-OH | 12 |
| E12 | H2N-IALILEPICCQ(Sar)RAA-OH | 13 |
| R13 | H2N-IALILEPICCQE(Sar)AA-OH | 14 |
| A14 | H2N-IALILEPICCQER(Sar)A-OH | 15 |
| A15 | H2N-IALILEPICCQERA(Sar)-OH | 16 |

### Buffers

Complement permissive GVBS⁺⁺ buffer comprising veronal buffered saline, 0.1% gelatin, 0.15 mM CaCl₂, and 1 mM MgCl₂ was prepared [12]. Complement inhibitory buffer GVBS⁻⁻ comprising veronal-buffered saline with 0.1% gelatin and 10 mM EDTA was prepared.

### Pooled Normal Human Serum (NHS)

Pooled normal human serum (NHS) was prepared as previously described [12]. Blood from at least 4 healthy human donors was collected in Vacutainer tubes without additives (red top). The blood was allowed to sit for 30 minutes at room temperature and 2 hours on ice so as to clot the blood and separate the serum. The sera were then pooled, aliquoted and frozen at −80° C.

### Hemolytic Assays of Complement Activity

For hemolytic complement assays, human red blood cells (RBCs) from type AB donors were purified, washed and standardized to 1×10⁹ cells/ml, as previously described [13]. Human sera from type 0 donors at a 20% final concentration was combined with 1 mM PIC1 or sarcosine variant peptides, and the volume was brought up to 0.15 ml with GVBS⁺⁺ and 0.5 ml RBCs. For factor B-depleted sera hemolytic assays, a final of 0.005% factor B depleted sera was incubated with 1 mM PIC1 or sarcosine variant peptides with 0.1 ml antibody-sensitized sheep red blood cells (EA) in a final volume of 0.75 ml GVBS⁺⁺. The samples were incubated for one hour at 37° C. Then, 1.0 ml of GVBS⁻⁻ was added to the factor B-depleted samples to stop the reaction. The samples were spun at 3,000 rpm for 5 minutes. The supernatant was then collected and read at 412 nm. An analysis was performed to represent the values as a percent of the positive control, which consists of human 0 sera and AB red blood cells in GVBS⁺⁺ buffer.

### MPO Activity Assay

PIC1 and sarcosine variants were diluted to 25 mg/ml and then serially titrated in a 96 well plate at a volume of 0.02 ml. Myeloperoxidase (MPO) was diluted to 20 μg/ml and 0.02 ml was added to the titrated peptides. TMB (3,3′,5,5′-tetramethylbenzidine) (0.1 ml) was added to each well for 2 minutes, followed by 0.1 ml of 2.5 N H₂SO₄ for another 2 minutes, and then read on a 96 well plate reader (BioTek) at 450 nm.

### C1q and MPO Binding Assays

An Immunlon-2 HB ELISA plate was coated with 1 μg/ml C1q or MPO in bicarbonate buffer overnight at 4° C. The plates were washed with PBST (phosphate buffered saline+0.1% Tween) and then blocked with 1% gelatin/PBS for 2 hours at room temperature. After washing, the plates were incubated with PA or sarcosine variant peptides starting at 2.5 mg/ml and then serially diluted in 1% gelatin/PBS for 1 hour at room temperature. After washing, the plates were probed with rabbit anti-PA (developed with Cocalico Biologicals, Reamstown, Pa.) at 1:1000 in 1% gelatin/PBS for 1 hour, room temperature, followed by a goat anti-rabbit HRP (Sigma Aldrich, St Louis, Mo.) at 1:1,000 in 1% gelatin/PBS for 1 hour at room temperature with a washing step in between. Wells were developed using TMB substrate solution, stopped using 1 N H₂SO₄, and read at 450 nm in a plate reader.

### MPO Heme Ring Oxidative Degradation

In a 96 well plate, 0.025 ml of 1.7 mg/ml MPO was combined with 0.00125 ml of 0.5% H₂O₂ and 3 mM PIC1 or sarcosine variant peptides in a total volume of 0.125 ml PBS and left to incubate at room temperature for 2 minutes. Using a 96 well plate reader, the wells were scanned for absorbance from 300-550 nm to generate curves reflecting the iron state in the MPO heme ring, as previously reported [14].

### Total Antioxidant Capacity Assay

The TAC (Total Antioxidant Capacity) Assay (Cell Biolabs, Inc. San Diego, Calif.) was used to measure the antioxidant capacity of the sarcosine variants based on the reduction of copper (II) to copper (I). The assay was performed according to the recommended kit protocol.

### NETosis Assay

NETosis assays with immune complexes were performed as previously described [9]. Briefly, normal human serum was stimulated with ovalbumin-antiovalbumin immune complexes in GVBS⁺⁺ for 30 minutes at 37° C. This mixture along with 0.05% H₂O₂ was then added to purified human neutrophils resuspended in RPMI with or without sarcosine variants (2 mM), allowing NETosis to occur. Quantitation of free DNA released from the neutrophils was performed using PicoGreen. Slides were stained with DAPI (Southern Biotech, Birmingham, Ala.). The following antibodies were used to visualize formation of NETs:
(i) mouse anti-elastase (Invitrogen, Carlsbad, Calif.) as a primary antibody with the secondary goat anti-mouse antibody Alexa Fluor 568 (Novus Biologicals, Centennial, Colo.) and
(ii) rabbit anti-histone H3 (Abcam, Cambridge Mass.) with goat anti-rabbit antibody Alexa Fluor 488 (Novus Biologicals).
   Fluorescence microscopy using an Olympus BX53 microscope was performed to visualize NETs.

### Statistical Analysis

Quantitative data were analyzed determining means, standard error (SEM), and Student's t-test [15] using Excel (Microsoft, Redmond, Wash.).

### Example 1: Peptide Solubility

To evaluate the influence of sarcosine residues on the solubility and biological function of the base peptide IALILEPICCQERAA (PA), peptide derivatives were synthesized with sarcosine residues substituted at all 15 positions. Also included is a peptide in which the vicinal cysteines at positions 9 and 10 (C9,C10) were replaced with a single sarcosine residue. The peptides are shown in Table 2. An assay of the water-solubility of each peptide was performed, with the results shown in Table 3. Substitution of sarcosine at positions A2, L3, I4, L5, I8, C9 and C9,10 resulted in peptides soluble in water. Due to their enhanced solubility in the absence of PEGylation, these peptides were selected for further evaluation of the various biological activities.

TABLE 3
Results of water-solubility assay of peptides

| | | SEQ | H2O |
|---|---|---|---|
| Name | Sequence | ID NO: | Soluble |
| PA-dPEG24 | H2N-IALILEPICCQERAA-dPEG24 | 19 | No |
| (PIC1) | | | |
| PA | H2N-IALILEPICCQERAA-OH | 1 | No |
| I1 | H2N-(Sar)ALILEPICCQERAA-OH | 2 | No |
| A2 | H2N-I(Sar)LILEPICCQERAA-OH | 3 | Yes |
| L3 | H2N-IA(Sar)ILEPICCQERAA-OH | 4 | Yes |
| I4 | H2N-IAL(Sar)LEPICCQERAA-OH | 5 | Yes |
| L5 | H2N-IALI(Sar)EPICCQERAA-OH | 6 | Yes |
| E6 | H2N-IALIL(Sar)PICCQERAA-OH | 7 | No |
| P7 | H2N-IALILE(Sar)ICCQERAA-OH | 8 | No |
| I8 | H2N-IALILEP(Sar)CCQERAA-OH | 9 | Yes |
| C9 | H2N-IALILEPI(Sar)CQERAA-OH | 10 | Yes |
| C10 | H2N-IALILEPIC(Sar)QERAA-OH | 11 | No |
| C9, 10 | H2N-IALILEPI(Sar)QERAA-OH | 29 | Yes |
| Q11 | H2N-IALILEPICC(Sar)ERAA-OH | 12 | No |
| E12 | H2N-IALILEPICCQ(Sar)RAA-OH | 13 | No |
| R13 | H2N-IALILEPICCQE(Sar)AA-OH | 14 | No |
| A14 | H2N-IALILEPICCQER(Sar)A-OH | 15 | No |
| A15 | H2N-IALILEPICCQERA(Sar)-OH | 16 | No |

### Example 2: Complement Inhibition Assays of Peptide Variants

An evaluation of the extent to which the peptide variants inhibit antibody-initiated complement activation was performed. The peptide variants were tested in the following two hemolytic assays: (i) an ABO incompatibility ex vivo assay and (ii) a classical pathway CH50-type assay in factor B-depleted sera.

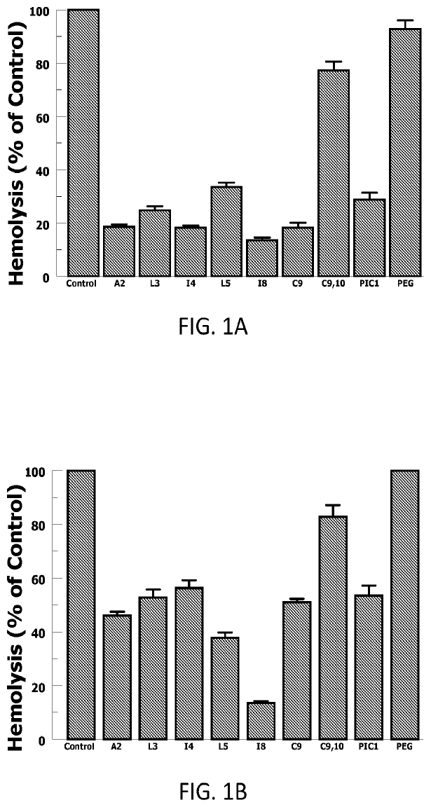

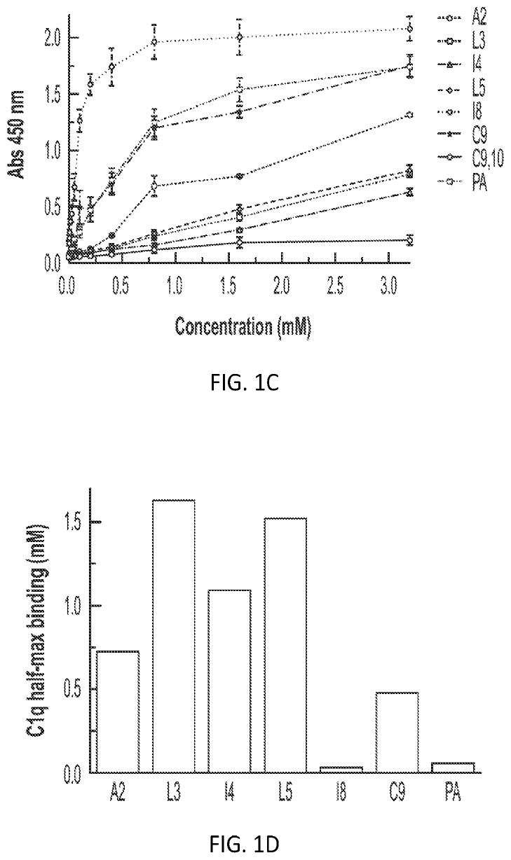

In the ABO incompatibility hemolytic assay, purified erythrocytes from a ‘type AB+’ donor were incubated with sera from a ‘type 0’ subject containing anti-A and anti-B antibodies; peptides were tested at 1.8 mM. The data are shown in FIG. 1A. Variants A2, 14, 18 and C9 each inhibited ABO incompatible hemolysis to a greater extent than did the PA-dPEG24 (PIC1) parent compound on an equimolar basis (P<0.015). The 18 variant decreased ABO hemolysis by 53% (P<0.002) more than PA-dPEG24. The C9,10 variant, however, showed minimal inhibition of ABO hemolysis.

A CH50-type hemolytic assay was then performed. with antibody-sensitized sheep erythrocytes, isolating the classical pathway by utilizing factor B-depleted sera; peptides were tested at 0.4 mM. The data are shown in FIG. 1B. In this assay the 18 variant demonstrated superior activity inhibiting hemolysis 75% (P<0.001) more than PA-dPEG24. Other peptides demonstrated similar inhibition of the classical complement pathway compared with PA-dPEG24 with the exception of C9,10, which again showed minimal activity.

An ELISA-type assay of peptide variant binding to C1q was then performed in which C1q was used as the capture substrate. Binding curves for each peptide are shown in FIG. 1C. From these binding curves, half-maximal binding concentrations were calculated, as shown in FIG. 1D. These binding curves and half-maximal binding calculations demonstrate that 18 and PA (the parent peptide) both yield superior binding to C1q as compared with the other peptides. However, PA has poor aqueous solubility, such that PA needs to be initially solubilized in DMSO and then diluted into an aqueous buffer. Higher concentrations of DMSO interfere with the detecting reagents resulting in a partial binding curve. The superior C1q binding of 18 correlates with superior inhibition of complement mediated hemolysis. Overall, the 18 variant shows superior inhibition of antibody-initiated complement activation and hemolysis compared with PA and other peptide variants.

### Example 3: Myeloperoxidase Inhibition and Binding

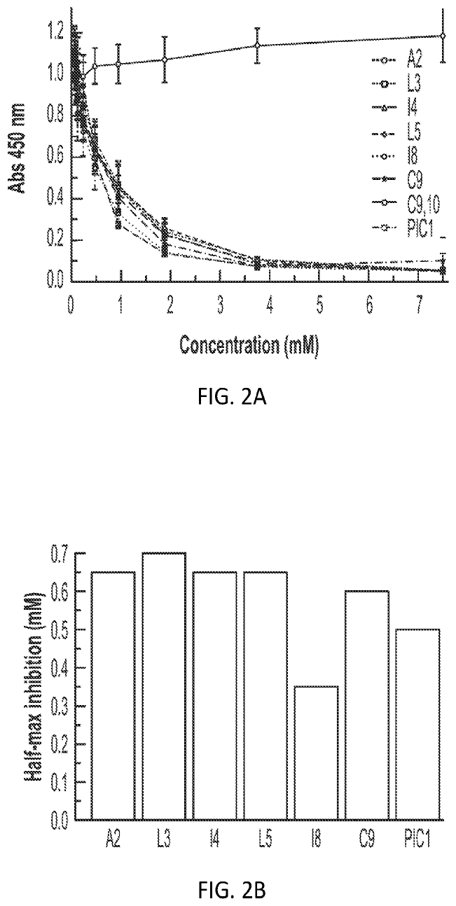

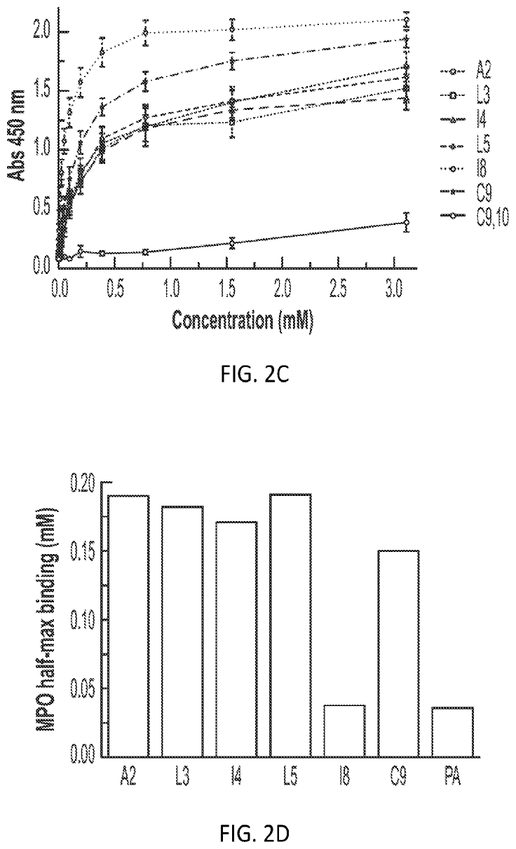

Inhibition of myeloperoxidase (MPO) activity when then tested in a TMB-based in vitro assay, as previously described for PA-dPEG24 [7]. In this assay, the variants were tested for MPO inhibition over a range of concentrations. The data is shown in FIG. 2A. Strong MPO inhibition was found for all variants with the exception of the no-cysteine variant (C9,10). Half-maximal inhibition values were calculated from the dose-response curves for each variant, with the data shown in FIG. 2B. There were measurable differences in MPO inhibition, with variant 18 again showing the greatest potency among the different variants.

A plate-based assay was performed to test the binding of the peptide variants to solid phased MPO. The binding curves are shown in FIG. 2C. MPO binding was identified for all variants, except for C9,10. Due to the near complete overlay of the 18 and PA curves, the PA curve is not shown in the graph. Half-maximal binding concentrations were calculated from these curves, with the results shown in FIG. 2D. 18 demonstrated superior binding to MPO, as compared with the other sarcosine variants. This result is consistent with the increased MPO inhibition identified by the dose-response data above.

### Example 4: Protection of the MPO Heme Ring from Degradation

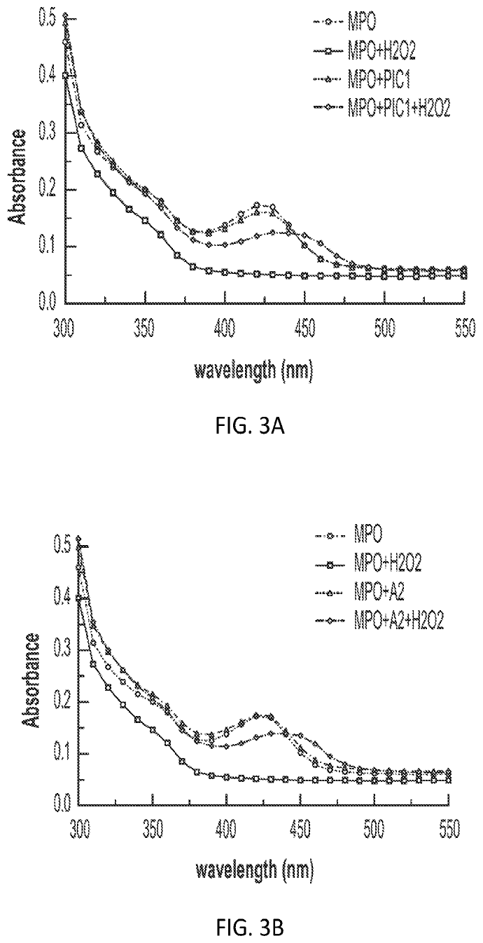

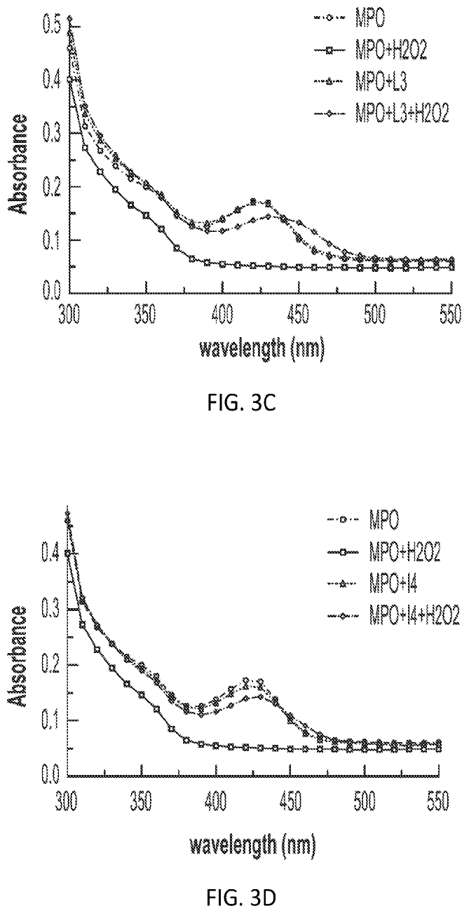

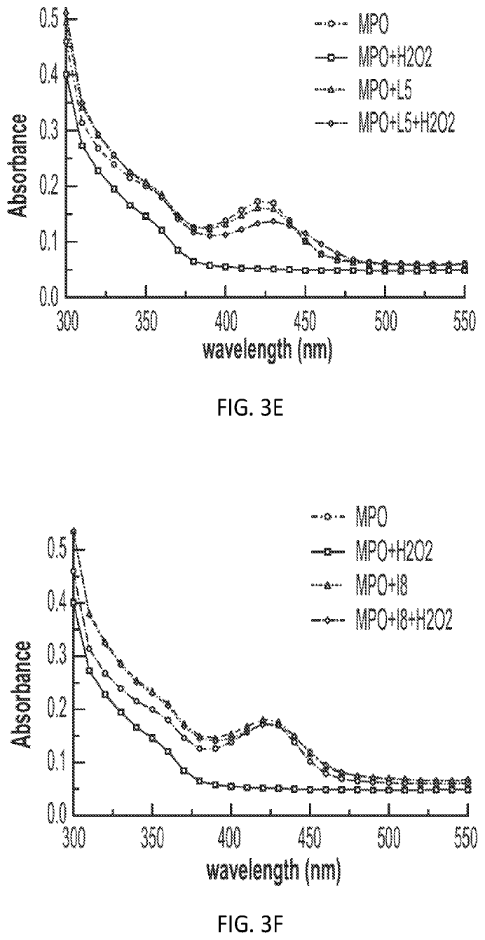

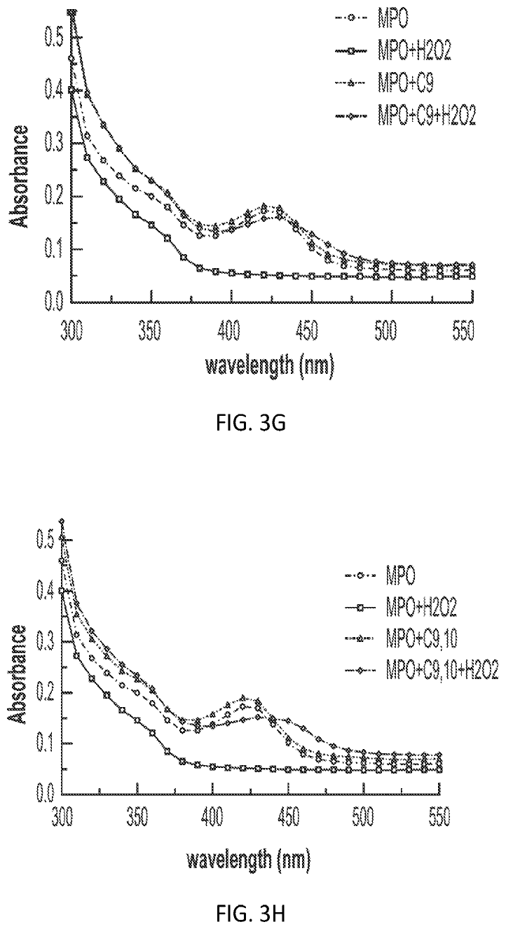

The ability of the variants to prevent degradation of the heme ring in the MPO molecule was then evaluated. Because MPO produces hypochlorous acid in the presence of chloride and hydrogen peroxide, the hypochlorous acid will degrade the heme ring, as previously shown for PA-dPEG24 [8]. Degradation of the heme ring can be evaluated via spectrometric measurements of absorption of wavelengths from 300 to 550 nm. Absorption measurements were conducted over this spectrum for all variants, with the data for each variant shown in FIGS. 3A-3H. All peptides showed some inhibition of heme ring degradation with near perfect preservation of the heme ring absorption spectrum for the 18 and C9 variants, as shown in FIGS. 3F and 3G. The heme ring preservation for these two variants was greater than that for the PA-dPEG24 peptide (FIG. 3A). The C9,10 variant also showed inhibition of heme ring degradation (FIG. 3H), suggesting a mechanism of protection other than inhibition of MPO peroxidase activity, which the C9,10 variant does not possess (as shown in FIG. 2A).

### Example 5: Antioxidant Capacity Assays

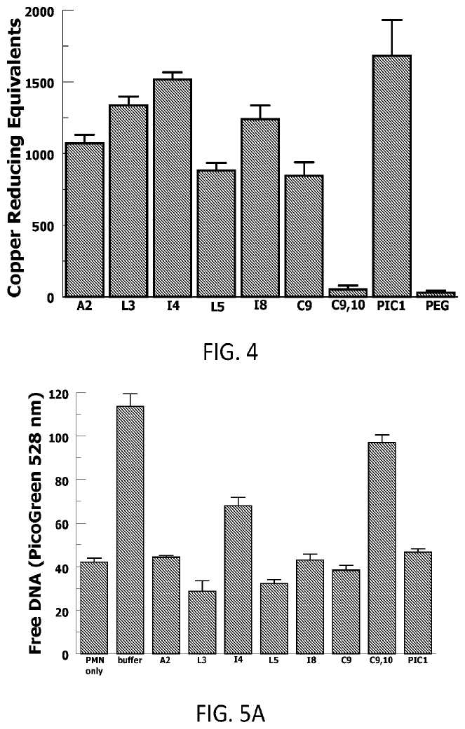

Antioxidant capability for the variants in a Total Antioxidant Capacity (TAC) assay was tested, as previously described for PA-dPEG24 [10]. Antioxidant activity was measured in copper reducing equivalents (CRE). Peptides were tested over a range of concentrations from 0.03-0.25 mM then compared to the standard. The results are shown in FIG. 4. The variants showed differing total antioxidant capacity, with L3, 14 and 18 demonstrating antioxidant capacity approaching that of PA-dPEG24 (PIC1). The C9,10 no-cysteine variant demonstrated no antioxidant capacity.

### Example 6: NETosis Inhibition

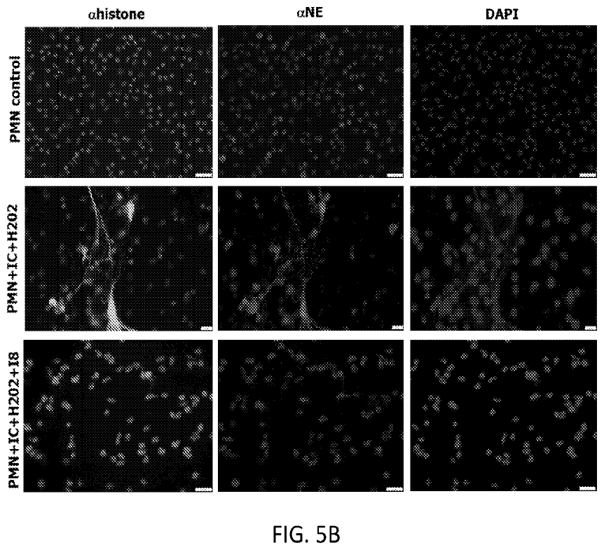

The peptide variants were tested for inhibition of NETosis. In this assay, purified human neutrophils were stimulated with normal human serum activated with ovalbumin-antiovalbumin immune complexes and hydrogen peroxide. Free DNA expressed from the neutrophils was then measured in a PicoGreen assay. The results are shown in FIG. 5A. In this assay multiple variants including A2, L3, L5, 18 and C9 demonstrated similar ability to inhibit NETosis compared with PA-dPEG24, as shown in the graph of FIG. 5A. 14 demonstrated a reduced ability to inhibit NETosis compared with PA-dPEG24 and C9,10 showed only slight inhibition relative to the negative control (buffer). These results show that many of the sarcosine variant peptides are able to inhibit NETosis to baseline values of free DNA from unstimulated neutrophils. Direct visualization of neutrophil extracellular trap (NET) formation by fluorescence microscopy was used to confirm the above NETosis results with the 18 variant. Human neutrophils were purified and stimulated on glass slides then stained with DAPI to visualize DNA and probed with anti-neutrophil elastase (αNE) and anti-histone H3 (αhistone), as previously described [9]. Unstimulated neutrophils showed no evidence of NETs (FIG. 5B, first row), neutrophils stimulated with ovalbumin-antiovalbumin immune complexes and hydrogen peroxide showed many NETs (FIG. 5B, second row) and neutrophils stimulated with immune complexes in the presence of PA-dPEG24 showed a dramatic reduction in NETs (FIG. 5B, third row). Fluorescence microscopy visualization of NETs confirmed the findings seen for quantitative measurements of free DNA.

The properties of the tested peptides are summarized in Table 4.

TABLE 4
Summary of Peptide Properties

| | PA- | | | | | | | C9, |
|---|---|---|---|---|---|---|---|---|
| Assay | dPEG24 | A2 | L3 | I4 | L5 | I8 | C9 | 10 |
| ABO C’ | +++ | ++++ | +++ | ++++ | +++ | ++++ | +++ | 0 |
| inhibition | | | | | | | | |
| Classical C’ | ++ | ++ | ++ | ++ | +++ | ++++ | ++ | 0 |
| inhibition | | | | | | | | |
| MPO inhibition | +++ | ++++ | +++ | +++ | +++ | ++++ | +++ | 0 |
| NETosis | +++ | +++ | ++++ | ++ | ++++ | +++ | +++ | + |
| inhibition | | | | | | | | |
| Heme ring | ++ | ++ | ++ | +++ | +++ | ++++ | +++ | ++ |
| protection | | | | | | | | |
| Total | ++++ | ++ | +++ | ++++ | ++ | +++ | ++ | 0 |
| Antioxidant Cap | | | | | | | | |

* previously published
ND = not done

An unanticipated finding is that the 18 variant is superior to the parent compound, PA-dPEG24, in most of the anti-inflammatory assays tested. Without wishing to be bound by theory, the C1q binding for 18 is slightly superior to that of PA-dPEG24 and may be driving enhanced inhibition of C1 activation.

Also unanticipated is that the C9 variant performed almost identically to the parent compound, PA-dPEG24, in terms of complement inhibition, despite the loss of one of the two cysteine amino acids. In the total antioxidant capacity assay, C9 had approximately half of the antioxidant capacity as PA-dPEG24, commensurate with having half as many cysteines. In the complement inhibition assays where no cysteines were present (variant C9,10) most, but not all, complement inhibition was lost. Host complement inhibitors are typically cysteine rich [17]. Together these findings suggest that the cysteines contribute to complement inhibition, but do not account for the entirety of complement inhibition for these peptides.

The following Materials and Methods section applies to Examples 7-11 below.

### Materials and Methods for Examples 7-11

### Ethics Statement

Blood from healthy donors was obtained with written consent under an Eastern Virginia Medical School IRB approved protocol, 02-06-EX 0216. Blood was used for the preparation of reagents: purified platelets and normal human sera.

### Reagents

PIC1 (IALILEPICCQERAA-dPEG24) was manufactured by PolyPeptide Group (San Diego, Calif.) to ≥95% purity verified by HPLC and mass spectrometry analysis. Lyophilized PIC1 was solubilized in a 0.05 M Histidine buffer pH 6.7. Platelet-sensitizing antibody was obtained from Tissue for Research LTD (Suffolk, United Kingdom) in the form of immune thrombocytopenic purpura (ITP) patient sera. PrestoBlue® Cell Viability Reagent was manufactured by Life Technologies (Eugene, Oreg.). PKH26 Red Fluorescent Cell Dye was manufactured by Sigma-Aldrich (St. Louis, Mo.).

### Buffers

Complement permissive GVBS⁺⁺ buffer comprising veronal buffered saline, 0.1% gelatin, 0.15 mM CaCl₂, and 1 mM MgCl₂ was prepared [12]. Complement inhibitory buffer GVBS⁻⁻ comprising veronal-buffered saline with 0.1% gelatin and 10 mM EDTA was prepared.

### Pooled Normal Human Serum (NHS)

Pooled normal human serum (NHS) was prepared as previously described [12].

### Rat Sera

Rat sera was purchased from Innovative Research (Peary Court, Novi, Mich.).

### Human Platelet Purification

Platelets were purified from multiple healthy human donors in order to ensure reproducibility across human donors. Peripheral blood (8 mL) from healthy volunteers was drawn directly into Vacutainer (BD, Franklin Lakes, N.J.) sterile collection tubes containing acid citrate dextrose (ACD) and used within 3 hours of collection. After adding additional 250 ul ACD, the blood was centrifuged at a sequence of 500×g, 100×g and 800×g for 5 minutes each at 22° C., with supernatant collected and transferred to a new tube after each spin. The purified platelets were then washed with GVBS⁻⁻, then saline, followed by a final resuspension in GBVS⁺⁺.

### Platelet Staining

For in vitro experiments, platelets were stained with PrestoBlue Cell Viability Reagent as per manufacturer's recommendations. For in vivo experiments, platelets were stained with PKH26 Red Fluorescent Cell Dye as per manufacturer's recommendations.

### In Vitro Complement and Platelet Studies

For experiments sensitizing platelets with human anti-platelet antibody, purified platelets were treated with 4% ITP sera for 15 minutes at 30° C. before adding 2% normal human complement sufficient sera and incubating for 30 minutes at 37° C. For samples containing PIC1, sera and PIC1 were pre-incubated for 5 minutes before combining with sensitized platelets. Samples were then spun and supernatant was removed for C5a analysis by ELISA. A C5a ELISA kit (R&D Systems) was used per manufacturer's instructions. The cells were washed once with GVBS⁻⁻ and once with water to lyse the platelets. The remaining cell pellet was treated with 25 mM methylamine for 1 hour at 37° C., spun, and the supernatant was analyzed for iC3b by ELISA. The ELISA for iC3b was performed as previously described, utilizing a goat anti-human C3 antibody (Complement Technology, Tyler, Tex.) for capture, a mouse anti-human iC3b antibody (Quidel, San Diego, Calif.) for probing, and a goat anti-mouse HRP (Sigma-Aldrich, St Louis, Mo.) for detection [30].

For experiments using rat sera, purified platelets were incubated with 5% Wistar rat sera at different time points (5 min, 20 min and 60 min) in a 37° C. bath. Thereafter, GVBS⁻⁻ was added to the mixture and this was spun at 500 g for 5 min at 22° C. After resuspending with 250 ul of PBS, PrestoBlue was added to the cells at a 1:10 dilution and incubated in a 37° C. water bath for 30 min. The cells were then plated and read on a microplate reader at 530 nm. With the addition of PIC1 to sera, there was a preincubation time of 5 minutes before addition of platelets.

### Animal Experiments

Male Wistar rats approximately 16 weeks old and 250 grams in weight were purchased (Hilltop Scottdale, Pa.) with indwelling intra jugular catheters in place. Rats were sedated with ketamine and acepromazine throughout the course of the experiment with monitoring of vital signs. PIC1 infusions were performed 30 seconds prior to transfusion with PKH26 stained human platelets. Blood samples were collected into K₂EDTA microtainer tubes (BD, Franklin Lakes, N.J., USA) from the animals prior to transfusion and then at 0.5, 2, 5, 20, 60 and 120 min after transfusion. Upon completion of the final blood draw, the animals were euthanized using Fatal Plus (Vortech Pharmaceuticals, Dearborn, Mich., USA). A necropsy was completed after euthanasia to collect organs for histopathology.

### Flow Cytometry

Flow cytometry was performed using a FACSCalibur flow cytometer (Becton Dickinson, Franklin Lakes, N.J., USA) with DXP 8 Color 488/637/407 upgrade (Cytek Development, Freemont, Calif., USA). The data was acquired using Cytek FlowJo CE version 7.5.110.7. Prior to analysis on the flow cytometry machine, whole rat blood was spun at 500×g for 5 min. The plasma platelet layer was removed and then diluted 1:10 with PBS. Approximately 20,000 events selected for platelet size were run on the FlowJo machine. Data was analyzed using FlowJo V10.4.2 (FlowJo LLC, Ashland, Oreg., USA).

### Stained Platelet Organ Histology

Liver and spleen were recovered after euthanasia, fixed in formalin and sectioned for histology slides. Slides were visualized with a fluorescence microscope.

### Statistical Analysis

Quantitative data were analyzed determining means, standard error (SEM), and Student's t-test using Excel (Microsoft Redmond, Wash.) [15].

### Example 7: Human Sera Complement Attack of Antibody-Sensitized Human Platelets

In order to elucidate the extent to which antibody-sensitized platelets can activate complement ex vivo experiments with purified human platelets and pooled normal human sera (NHS) were performed as follows. Without wishing to be bound by theory, in clinical medicine platelet refractoriness is commonly associated with the presence of anti-platelet antibodies. In these experiments ITP sera antibodies were utilized for sensitizing the platelets.

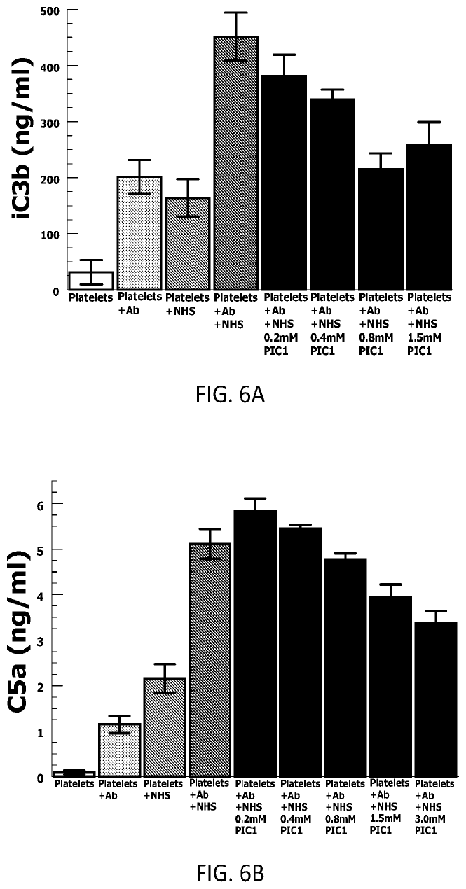

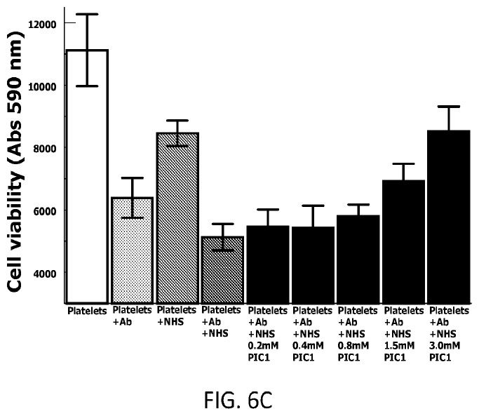

Human platelets were incubated with sensitizing antibody, NHS, or both. Increasing concentrations of PA-dPEG24 (PIC1) were added in the presence of sensitizing antibody and NHS. Complement attack of the platelets and cell viability were assayed. C3 is the central component of the complement activation cascades. The C3 fragment iC3b is an opsonin that contributes to phagocytosis. C3 activation on the platelet surface was assayed by iC3b ELISA of stripped platelet membrane bound proteins, with the data shown in FIG. 6A. The experiment involved an iC3b assay of (i) platelets alone, (ii) platelets with sensitizing antibody, (iii) platelets with NHS, (iv) platelets with both sensitizing antibody and NHS, and (v) platelets with sensitizing antibody, NHS, and increasing amounts of PIC1. The results indicate antibody-initiated classical complement pathway-mediated attack. Increasing concentrations of PA-dPEG24 decreased iC3b opsonization of the platelets in a dose-dependent manner. At 0.8 mM PA-dPEG24, iC3b was bound to platelets at a level similar to that of the background level measured for sensitizing antibody alone.

Plasma recovered from these experiments was then assayed for the pro-inflammatory anaphylatoxin C5a by ELISA. The data is shown in FIG. 6B. As with bound iC3b, C5a generation was markedly increased for platelets incubated in the presence of sensitizing antibody and NHS compared with either alone. Increasing concentrations of PA-dPEG24 dose-dependently inhibited C5a generation. Significant inhibition of C5a generation occurred at the 1.5 mM (P=0.028) and 3.0 mM (44% decrease, P=0.006).

Cell viability of the platelets was measured by utilizing the vital dye, Presto Blue. The data is shown in FIG. 6C. Platelet viability dose-dependently increased with increasing concentrations of PA-dPEG24 in the presence of sensitizing antibody and NHS, as compared with platelets incubated with sensitizing antibody and NHS only. Significant increases in platelet viability occurred at the 1.5 mM (P=0.031) and 3.0 mM (53% increase, P=0.003) concentrations of PA-dPEG24. These results suggest that anti-platelet antibodies may activate complement, leading to opsonization, anaphylatoxin generation and decreased cell viability. These effects can be largely reversed in the presence of a classical complement pathway inhibitor.

### Example 8: Rat Sera Complement Attack of Antibody-Sensitized Human Platelets

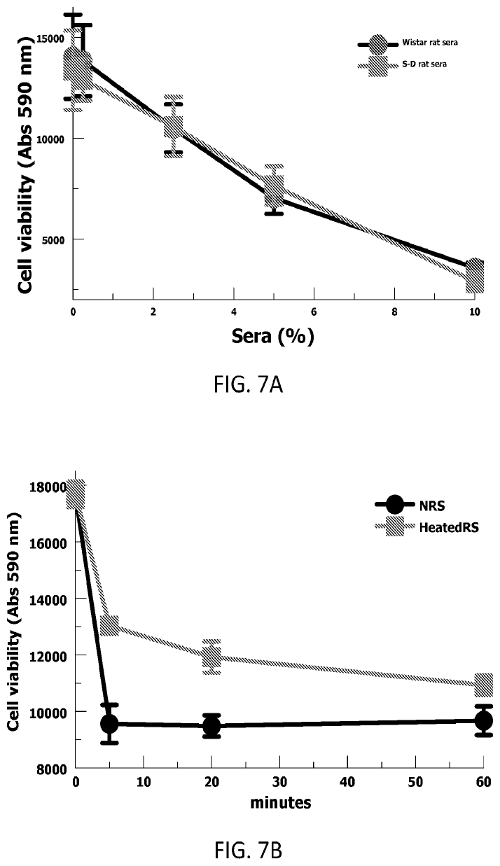

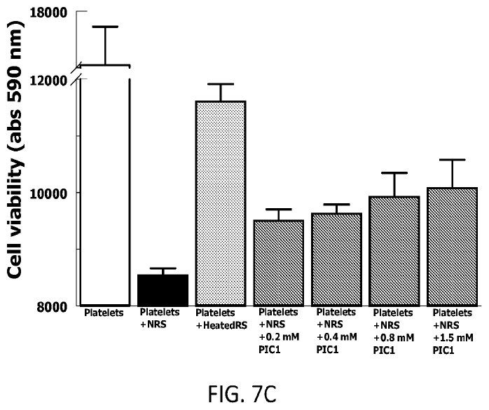

In order to translate our ex vivo findings into an animal model of platelet refractoriness, the extent to which human platelets activate complement in rat sera was assayed. A previously developed allogeneic erythrocyte transfusion model of acute hemolytic transfusion reaction (AHTR) was used for the assay [29]. In this model, natural antibodies preexisting in Wistar rat plasma were directed against human erythrocytes, leading to complement-mediated hemolysis as well as extravascular removal of transfused human erythrocytes. Also in this model, complement inhibition increased the number of circulating transfused erythrocytes for a period of time [29, 31]. In order to evaluate the extent to which rat sera could decrease human platelet viability, human platelets were incubated in increasing concentrations of rat sera from Wistar or Sprague Dawley rats. The data is shown in FIG. 7A. Cell viability was assayed with the vital dye Presto Blue. As rat sera concentration increased, the number of viable human platelets dramatically decreased such that after incubation in 10% sera for 30 minutes, only 25% or fewer platelets were viable (P<0.02).

Wistar rat sera complement-mediated killing of human platelets was then tested in a time course experiment. The data are shown in FIG. 7B. Heat-inactivated rat sera was used as a control to isolate the complement-mediated effect on platelet viability. The number of viable platelets was assayed with Presto Blue in 5% complement-sufficient rat sera. The number of viable platelets decreased by nearly 50% in the first 5 minutes, and remained steady at later time points. These data suggest that the complement-mediated decrease in platelet viability occurs very quickly, likely reflecting complement-mediated cell lysis. At five minutes, a two-fold decrease in platelet viability was seen for human platelets in complement-sufficient rat sera, as compared with heat-inactivated rat sera (P=0.01). These data demonstrate that the complement system in rat sera can rapidly kill human platelets.

In order to evaluate the extent to which rat sera killing of human platelets is mediated by the classical pathway of complement, we utilized PA-dPEG24. In vitro experiments with human platelets and rat sera were performed as described above including incubations in sera with increasing concentrations of PA-dPEG24. The data are shown in FIG. 7C. Viability was assayed with Presto Blue vital dye, with results showing a dose-dependent improvement in platelet viability with increasing doses of PA-dPEG24. PA-dPEG24 improved survival for human platelets in rat sera for all doses tested compared to control, platelets in normal rat sera (P<0.017). Together these data suggest that human platelets in rat sera will experience decreased viability much of which can be reversed by complement inactivation or inhibition.

### Example 9: Flow Cytometry Assays of PKH26 Stained Platelets

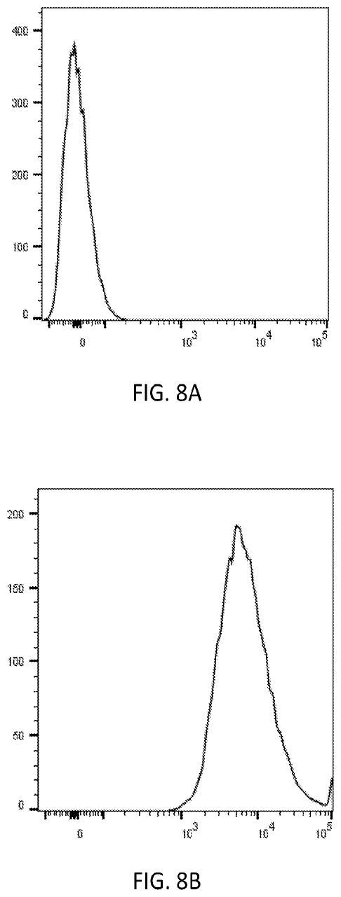

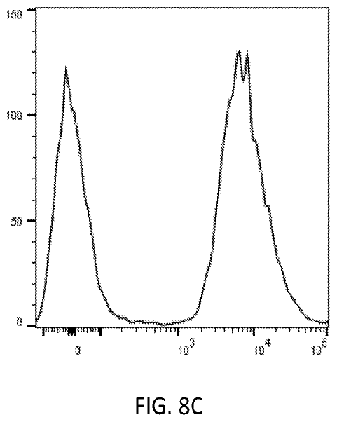

A previously reported method for staining platelets with PKH26 was optimized and used to assay transfused platelets in rat blood samples [32]. PKH26 has been used to stain human erythrocytes prior to transfusion into Wistar rats, followed by measuring the persistence of erythrocytes in circulation by flow cytometry [31]. As shown in the data of FIG. 8A, unstained purified human platelets demonstrated a typical distribution with low background signal by flow cytometry at 530/30 nm. In FIG. 8B, PKH26 stained purified human platelets showed peak signal intensity approximately 10⁴ greater than background. The stained and unstained platelets were mixed in a 1:1 ratio. As shown in FIG. 8C, the flow cytometry histogram showed good separation of the peaks and similar area under the curve. These results indicate that the optimized method of PKH26 staining of human platelets yields reliable flow cytometry data after transfusion.

### Example 10: Pilot Transfusion of PKH26 Stained Platelets

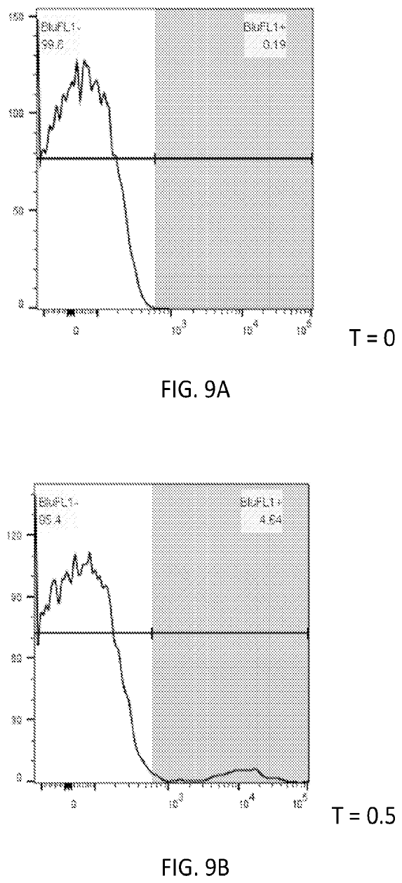

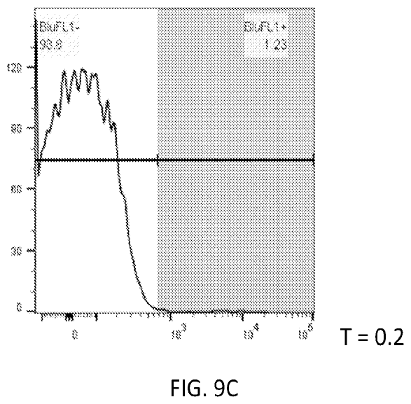

A pilot study to evaluate whether PKH26 stained human platelets could be measured in post-transfusion blood samples was performed on six rats with indwelling jugular catheters. Three rats received 1×10⁸ PKH26 stained human platelets IV. Three rats received sham transfusions. Blood samples were obtained prior to transfusion (T=0) and at 0.5, 2, 5, 20, 60 and 120 minutes after transfusion. Blood was collected into pediatric K₂EDTA vials then diluted with saline and analyzed by flow cytometry. Only a minimal stained platelet signal was detected for the T=0 sample, as shown in FIG. 9A. At 0.5 minutes after transfusion the PKH26 stained platelets were detectable at the correct fluorescence intensity, as shown in FIG. 9B. The stained platelets measured approximately 5% of the total number of platelets in the blood samples at the 0.5 minute time point for each of the three transfused rats. At two minutes after transfusion the signal from the PKH26 stained platelets was decreased, but still measurable above background, as shown in FIG. 9C. At later time points, the flow cytometry detection of stained platelets in the blood samples continued to decline.

The data indicate that transfusions with 1×10⁸PKH26 stained human platelets IV can yield detectable signal by flow cytometry. The number of transfused human platelets that remain in the rat blood stream decreased rapidly over time, consistent with immunological incompatibility.

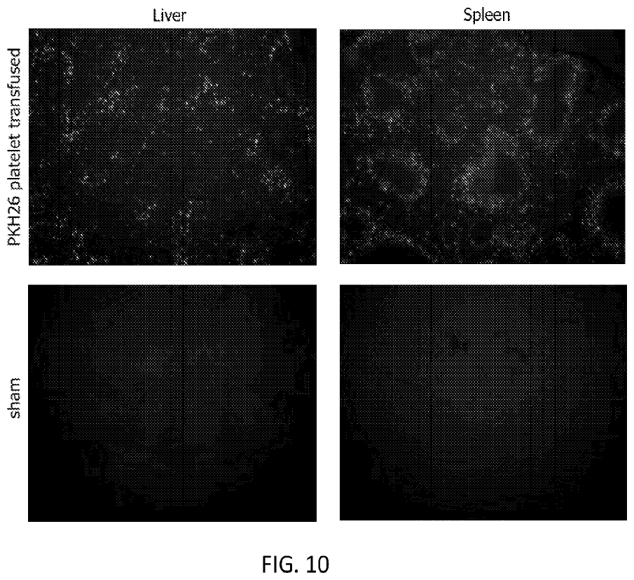

It was then evaluated whether the PKH26 transfused platelets could be identified in the liver and spleen of the rats by fluorescence microscopy. The liver and spleen were recovered after euthanasia, fixed with formalin and sectioned by standard methods. The PKH26 stained platelets were easily visualized in the liver (as shown in FIG. 10A) as well as in the spleen (as shown in FIG. 10B), where distribution in the red pulp was evident. These results suggest a robust extravascular removal of platelets in this model.

### Example 11: Complement Inhibition and Transfusion of Incompatible Platelets

A classical complement pathway inhibitor PA-dPEG24 was used to evaluate whether inhibition of the classical complement pathway could affect the number of transfused incompatible platelets in the blood stream. PA-dPEG24 can increase the numbers of incompatible transfused erythrocytes in the bloodstream in a rat model of AHTR. As shown in FIG. 6C, PA-dPEG24 can increase survival of human platelets in rat sera.

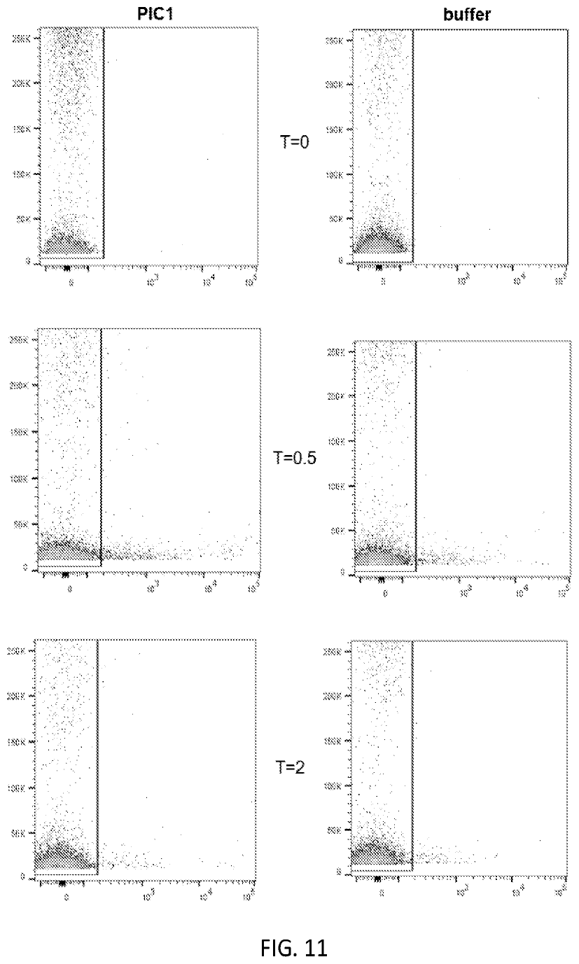

Transfusions were performed with PKH26 stained human platelets as described above after infusion of (i) PA-dPEG24 or (ii) a saline vehicle control. FIG. 11 shows representative flow cytometry measurements selected for platelets at 0, 0.5 and 2 minutes after transfusion for rats treated with PA-dPEG24 (PIC1) or saline control. The unstained native rat platelets are to the left of the vertical line, and the stained transfused platelets are to the right. At 0.5 minutes after transfusion, increased numbers of stained transfused platelets are seen for the PA-dPEG24 treated animal, as compared with the animal receiving vehicle. This difference is not noticeable at two minutes.

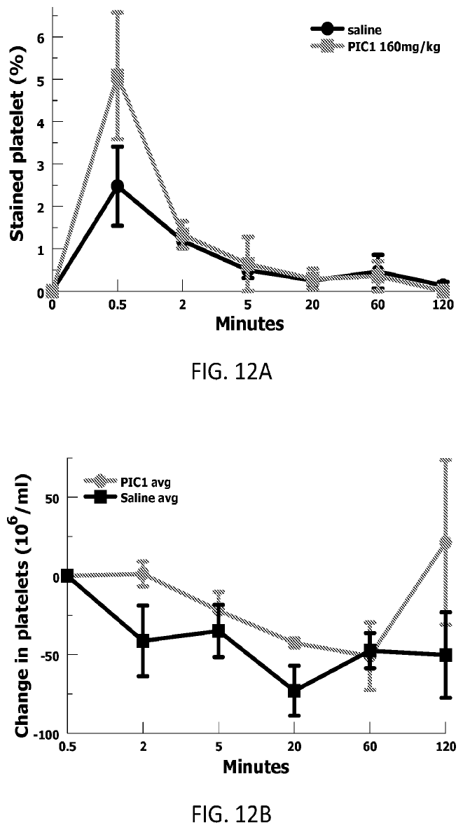

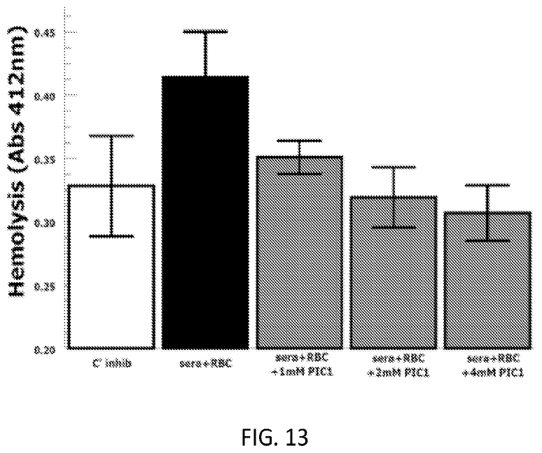

The compiled data for these experiments in FIG. 12A shows a 2-fold increase (P=0.05) in transfused platelets at 0.5 minutes for animals receiving PA-dPEG24 compared with control, with no significant difference in the amount of transfused platelets at later time points. Total platelet counts for rats transfused with unstained platelets treated with PIC1 or saline control (as shown in FIG. 12B) were evaluated in order to evaluate if similar trends could be determined for unstained platelets. Platelet counts were measured for each blood sample by Antech Diagnostics (Chesapeake, Va.). Decreases in platelet counts were calculated for each time point compared with the maximal platelet count immediately after transfusion at 30 seconds. Statistically significant greater decreases in the platelet count were found for saline-treated versus PA-dPEG24-treated animals at two minutes (P=0.04) and 20 minutes (P=0.03). These results suggest that human platelets transfused into Wistar rats can yield a platelet-refractory phenotype and that transfused platelet survival can be briefly increased with classical complement inhibition.

### Example 12: PIC1 for Treatment of a Delayed Hemolytic Transfusion Reaction

A 14-year old with sickle cell disease received a pRBC (packed Red Blood Cell) transfusion for vaso-occlusive disease and acute chest syndrome. Eight days after transfusion, she suffered worsening extremity pain that was followed by new onset of fever, hypertension and respiratory decompensation. Her hemoglobin decreased from 7.6 to 5.0 g/dL overnight consistent with a delayed hemolytic transfusion reaction. She subsequently survived an eight-day ICU stay requiring multiple pRBC transfusions and receiving multiple immunomodulators including methylprednisolone, IVIg (intravenous immunoglobulin), eculizumab, rituximab and tocilizumab. In order to better understand her disease process, the patient's erythrocytes and plasma were analyzed in hemolytic assays in the presence or absence of a classical complement pathway inhibitor, PIC1 (PA-dPEG24).

Discarded de-identified blood was obtained from routine medical management blood draws from the patient prior to treatment with eculizumab. Hemolytic complement assays utilizing the patient's plasma and erythrocytes in complement permissive buffer (GVBS⁺⁺) or complement inhibitory buffer (GVBS⁻⁻) were performed in the presence of increasing concentrations of PA-dPEG24. Complement permissive GVBS⁺⁺ buffer is veronal-buffered saline with 0.1% gelatin, 0.15 mM CaCl₂, and 1 mM MgCl₂ [11]. Complement inhibitory buffer GVBS⁻⁻ is a veronal-buffered saline with 0.1% gelatin and 10 mM EDTA. Approximately 5×10⁷ washed erythrocytes were incubated with 20% plasma in a reaction volume of 0.15 ml for 1 hour at 37° C.

Erythrocytes were sedimented by centrifugation at 1,500×g for 5 minutes and the supernatant was then recovered. The supernatant was measured on a Bio Tek Synergy HT plate reader spectrophotometer for absorption at 412 nm. Hemolysis was measured by quantitation of free hemoglobin on the spectrophotometer at 412 nm.

The patient's plasma caused hemolysis of her erythrocytes in complement permissive buffers, demonstrating complement-mediated hemolysis (FIG. 1). Addition of PIC1 showed a statistically significant dose-dependent decrease of hemolysis. At higher doses. PIC1 inhibited hemolysis to the background signal.

For this patient with sickle cell disease and DHTR, complement-mediated hemolysis of her erythrocytes by her plasma was demonstrated ex vivo. The complement-mediated hemolysis was completely blocked at higher doses with a classical complement pathway inhibitor, PIC1.

The above data show that a classical complement pathway inhibitor can be used to treat active hemolysis in a patient suffering a delayed hemolytic transfusion reaction. Additionally, this data demonstrates that complement-mediated hemolysis plays a significant role in DHTR pathogenesis.

### REFERENCES

1. Matsui S M, Kiang D, Ginzton N, Chew T, and Geigenmuller-Gnirke U (2001) Molecular biology of astroviruses: selected highlights. Novartis Found Symp 238: 219-233; discussion 233-216.
2. Sebire N J, Malone M, Shah N, Anderson G, Gaspar H B, et al. (2004) Pathology of astrovirus associated diarrhoea in a paediatric bone marrow transplant recipient. J Clin Pathol 57: 1001-1003.
3. Bonaparte R S, Hair P S, Banthia D, Marshall D M, Cunnion K M, et al. (2008) Human astrovirus coat protein inhibits serum complement activation via C1, the first component of the classical pathway. J Virol 82: 817-827.
4. Frank M M, Miletic V D, and Jiang H (2000) Immunoglobulin in the control of complement action. Immunol Res 22: 137-146.
5. Gronemus J Q, Hair P S, Crawford K B, Nyalwidhe J O, Cunnion K M, et al. (2010) Potent inhibition of the classical pathway of complement by a novel C1q-binding peptide derived from the human astrovirus coat protein. Mol Immunol 48: 305-313.
6. Sharp J A, Hair P S, Pallera H K, Kumar P S, Mauriello C T, et al. (2015) Peptide Inhibitor of Complement C1 (PIC1) Rapidly Inhibits Complement Activation after Intravascular Injection in Rats. PLoS One 10: e0132446.
7. Hair P S, Sass L A, Krishna N K, and Cunnion K M (2017) Inhibition of Myeloperoxidase Activity in Cystic Fibrosis Sputum by Peptide Inhibitor of Complement C1 (PIC1). PLoS One 12: e0170203.
8. Hair P S, Cunnion K M, and Krishna N K (2017) Peptide Inhibitor of Complement C1 Inhibits the Peroxidase Activity of Hemoglobin and Myoglobin. Int J Pept 2017: 9454583.
9. Hair P S, Enos A I, Krishna N K, and Cunnion K M (2018) Inhibition of Immune Complex Complement Activation and Neutrophil Extracellular Trap Formation by Peptide Inhibitor of Complement C1. Front Immunol 9: 558.
10. Gregory Rivera M, Hair P S, Cunnion K M, and Krishna N K (2018) Peptide Inhibitor of Complement C1 (PIC1) demonstrates antioxidant activity via single electron transport (SET) and hydrogen atom transfer (HAT). PLoS One 13: e0193931.
11. Hair P S, Rivera M G, Enos A I, Pearsall S E, Sharp J A, et al. (2017) Peptide Inhibitor of Complement C1 (PIC1) Inhibits Growth of Pathogenic Bacteria. International Journal of Peptide Research and Therapeutics DOI 101007/s10989-017-9651-z.
12. Cunnion K M, Lee J C, and Frank M M (2001) Capsule production and growth phase influence binding of complement to Staphylococcus aureus. Infect Immun 69: 6796-6803.
13. Mauriello C T, Pallera H K, Sharp J A, Woltmann J L, Jr., Qian S, et al. (2013) A novel peptide inhibitor of classical and lectin complement activation including ABO incompatibility. Mol Immunol 53: 132-139.
14. Maitra D, Shaeib F, Abdulhamid I, Abdulridha R M, Saed G M, et al. (2013) Myeloperoxidase acts as a source of free iron during steady-state catalysis by a feedback inhibitory pathway. Free Radic Biol Med 63: 90-98.
15. Carlin J B and Doyle L W (2001) Statistics for clinicians: 4: Basic concepts of statistical reasoning: hypothesis tests and the t-test. J Paediatr Child Health 37: 72-77.
16. Moretto A, Crisma M, Kaptein B, Broxterman Q B, and Toniolo C (2006) N-methylation of N(alpha)-acylated, fully C(alpha)-methylated, linear, folded peptides: synthetic and conformational aspects. Biopolymers 84: 553-565.
17. Merle N S, Church S E, Fremeaux-Bacchi V, and Roumenina L T (2015) Complement System Part I—Molecular Mechanisms of Activation and Regulation. Front Immunol 6: 262.
18. Stanworth S J, Navarrete C, Estcourt L, and Marsh J. Platelet refractoriness—practical approaches and ongoing dilemmas in patient management. Br J Haematol 2015; 171: 297-305.
19. Slichter S J, Davis K, Enright H, Braine H, Gernsheimer T, Kao K J, Kickler T, Lee E, McFarland J, McCullough J, Rodey G, Schiffer C A, and Woodson R. Factors affecting posttransfusion platelet increments, platelet refractoriness, and platelet transfusion intervals in thrombocytopenic patients. Blood 2005; 105: 4106-14.
20. Jackman R P, Lee J H, Pei R, Bolgiano D, Lebedeva M, Slichter S J, and Norris P J. C1q-binding anti-HLA antibodies do not predict platelet transfusion failure in Trial to Reduce Alloimmunization to Platelets study participants. Transfusion 2016; 56: 1442-50.
21. Trial to Reduce Alloimmunization to Platelets Study G. Leukocyte reduction and ultraviolet B irradiation of platelets to prevent alloimmunization and refractoriness to platelet transfusions. N Engl J Med 1997; 337: 1861-9.
22. Pavenski K, Freedman J, and Semple J W. HLA alloimmunization against platelet transfusions: pathophysiology, significance, prevention and management. Tissue Antigens 2012; 79: 237-45.
23. Moncharmont P. Platelet component transfusion and alloimmunization: Where do we stand? Transfus Clin Biol 2018:25: 172-8.
24. Forest S K and Hod E A. Management of the Platelet Refractory Patient. Hematol Oncol Clin North Am 2016; 30: 665-77.
25. Liu W, Wu D, Hu T, and Ye B. Efficiency of treatment with rituximab in platelet transfusion refractoriness: a study of 7 cases. Int J Clin Exp Med 2015; 8: 14080-4.
26. Bordin J O, Bardossy L, and Blajchman M A. Experimental animal model of refractoriness to donor platelets: the effect of plasma removal and the extent of white cell reduction on allogeneic alloimmunization. Transfusion 1993; 33: 798-801.
27. Muench M O, Heitman J W, Inglis H, Fomin M E, Marschner S, Goodrich R P, Norris P J, and Jackman R P. Reduced alloimmunization in mice following repeated transfusion with pathogen-reduced platelets. Transfusion 2016; 56: 1419-29.
28. Sharp J A, Whitley P H, Cunnion K M, and Krishna N K. Peptide inhibitor of complement cl, a novel suppressor of classical pathway activation: mechanistic studies and clinical potential. Front Immunol 2014:5: 406.
29. Kumar P S, Pallera H K, Hair P S, Rivera M G, Shah T A, Werner A L, Lattanzio F A, Cunnion K M, and Krishna N K. Peptide inhibitor of complement C1 modulates acute intravascular hemolysis of mismatched red blood cells in rats. Transfusion 2016; 56: 2133-45.
30. Kumar P S, Mauriello C T, Hair P S, Rister N S, Lawrence C, Raafat R H, and Cunnion K M. Glucose-based dialysis fluids inhibit innate defense against Staphylococcus aureus. Mol Immunol 2015:67: 575-83.
31. Shah T A, Mauriello C T, Hair P S, Sharp J A, Kumar P S, Lattanzio F A, Werner A L, Whitley P H, and Maes L A, Cunnion K M, Krishna N K. Complement inhibition significantly decreases red blood cell lysis in a rat model of acute intravascular hemolysis. Transfusion 2014; 54: 2892-900.
32. Rand M L, Wang H, Mody M. Chu I, Treutiger I, Nguyen A, Packham M A, and Freedman J. Concurrent measurement of the survival of two populations of rabbit platelets labeled with either two PKH lipophilic dyes or two concentrations of biotin. Cytometry 2002; 47: 111-7.
33. Pirenne F and Yazdanbakhsh K. How I safely transfuse patients with sickle-cell disease and manage delayed hemolytic transfusion reactions. Blood. 2018; 131(25):2773-2781.
34. Silvergleid A J. Hemolytic transfusion reactions. Up to Date. Accessed 31 Dec. 2018.
35. Zimring J C and Spitalnik S L. Pathobiology of transfusion reactions. Annu. Rev. Pathol. Mech. Dis. 2015; 10:83-110.

All references, including publications, patent applications, and patents, cited herein are hereby incorporated by reference to the same extent as if each reference were individually and specifically indicated to be incorporated by reference and were set forth in its entirety herein.

1. A method for inhibiting an immune response to transfused platelets in a subject, said method comprising the steps of:
a) administering a classical complement pathway inhibitor to a subject in need thereof; and
b) transfusing platelets to the subject.

2. A method for inhibiting refractoriness to platelets in an alloimmunized subject, comprising the steps of:
a) treating platelets with a classical complement pathway inhibitor; and
b) transfusing the treated platelets to the subject.

3. A method of preventing platelet refractoriness in a subject receiving platelets from an antigenically mismatched donor, the method comprising administering a classical complement pathway inhibitor to the subject before the platelets are transfused to the subject.

4. A method of preventing platelet refractoriness in a subject receiving platelets from an antigenically mismatched donor, the method comprising the steps of:
a) treating platelets with a classical complement pathway inhibitor; and
b) transfusing the treated platelets to the subject.

5. The method of claim 1, wherein the method is effective to increase survival of the transfused platelets in the subject.

6. The method of claim 1, wherein the method is effective to reduce complement-mediated attack of the transfused platelets in the subject.

7. The method of claim 1, wherein the method is effective to increase survival of the transfused platelets in the subject.

8. The method of claim 1, wherein the subject is human.

9. The method of claim 1, wherein the complement mediated inhibitor is a PIC1 peptide.

10. The method of claim 9, wherein the PIC1 peptide comprises an amino acid sequence at least 85% identical to any one of SEQ ID NOS: 1-45.

11. The method of claim 10, wherein the PIC1 peptide comprises an amino acid sequence at least 85% identical to any one of SEQ ID NOS: 3, 4, 5, 6, 9, 10, 19, and 29.

12. The method of claim 11, wherein the PIC1 peptide comprises the amino acid sequence of SEQ ID NO: 3.

13. The method of claim 11, wherein the PIC1 peptide comprises the amino acid sequence of SEQ ID NO: 4.

14. The method of claim 11, wherein the PIC1 peptide comprises the amino acid sequence of SEQ ID NO: 5.

15. The method of claim 11, wherein the PIC1 peptide comprises the amino acid sequence of SEQ ID NO: 6.

16. The method of claim 11, wherein the PIC1 peptide comprises the amino acid sequence of SEQ ID NO: 9.

17. The method of claim 11, wherein the PIC1 peptide comprises the amino acid sequence of SEQ ID NO: 10.

18. The method of claim 11, wherein the PIC1 peptide comprises the amino acid sequence of SEQ ID NO: 19.

19. The method of claim 11, wherein the PIC1 peptide comprises the amino acid sequence of SEQ ID NO: 29.

20. A blood product for transfusion into a recipient, wherein the blood product comprises platelets and a classical complement pathway inhibitor.

21. The blood product of claim 20, wherein the complement mediated inhibitor is a PIC1 peptide.

22. The blood product of claim 21, wherein the PIC1 peptide comprises an amino acid sequence at least 85% identical to any one of SEQ ID NOS: 3, 4, 5, 6, 9, 10, 19, and 29.

23. The blood product of claim 22, wherein the PIC1 peptide comprises the amino acid sequence of any one of SEQ ID NOS: 3, 4, 5, 6, 9, 10, 19, and 29.

24. The blood product of claim 23, wherein the PIC1 peptide comprises the amino acid sequence of SEQ ID NO: 3.

25. The blood product of claim 23, wherein the PIC1 peptide comprises the amino acid sequence of SEQ ID NO: 4.

26. The blood product of claim 23, wherein the PIC1 peptide comprises the amino acid sequence of SEQ ID NO: 5.

27. The blood product of claim 23, wherein the PIC1 peptide comprises the amino acid sequence of SEQ ID NO: 6.

28. The blood product of claim 23, wherein the PIC1 peptide comprises the amino acid sequence of SEQ ID NO: 9.

29. The blood product of claim 23, wherein the PIC1 peptide comprises the amino acid sequence of SEQ ID NO: 10.

30. The blood product of claim 23, wherein the PIC1 peptide comprises the amino acid sequence of SEQ ID NO: 19.

31. The blood product of claim 23, wherein the PIC1 peptide comprises the amino acid sequence of SEQ ID NO: 29.

32. A method of treating delayed hemolytic transfusion reaction (DHTR) in a subject comprising administering a therapeutically effective amount of a classical complement pathway inhibitor to the subject.

33. The method of claim 32, wherein the classical complement pathway inhibitor is administered parenterally.

34. The method of claim 32, wherein the subject is human.

35. The method of claim 32, wherein the complement mediated inhibitor is a PIC1 peptide.

36. The method of claim 35, wherein the PIC1 peptide comprises an amino acid sequence at least 85% identical to any one of SEQ ID NOS: 3, 4, 5, 6, 9, 10, 19, and 29.

37. The method of claim 36, wherein the PIC1 peptide comprises the amino acid sequence of any one of SEQ ID NOS: 3, 4, 5, 6, 9, 10, 19, and 29.

38. The method of claim 37, wherein the PIC1 peptide comprises the amino acid sequence of SEQ ID NO: 3.

39. The method of claim 37, wherein the PIC1 peptide comprises the amino acid sequence of SEQ ID NO: 4.

40. The method of claim 37, wherein the PIC1 peptide comprises the amino acid sequence of SEQ ID NO: 5.

41. The method of claim 37, wherein the PIC1 peptide comprises the amino acid sequence of SEQ ID NO: 6.

42. The method of claim 37, wherein the PIC1 peptide comprises the amino acid sequence of SEQ ID NO: 9.

43. The method of claim 37, wherein the PIC1 peptide comprises the amino acid sequence of SEQ ID NO: 10.

44. The method of claim 37, wherein the PIC1 peptide comprises the amino acid sequence of SEQ ID NO: 19.

45. The method of claim 37, wherein the PIC1 peptide comprises the amino acid sequence of SEQ ID NO: 29.
